(19) United States
(12) Patent Application Publication
(10) Pub. No.: US 2025/0345619 A1
(43) Pub. Date: Nov. 13, 2025

(54) CARDIAC TREATMENT SYSTEM

(71) Applicant: Maxwell Biomedical, Inc., San Diego, CA (US)

(72) Inventors: Jeffrey PETERS, Excelsior, MN (US); Stephen BOLEA, Watertown, MN (US); Dave SERDAR, San Diego, CA (US); Charles STEADERMAN, San Diego, CA (US); Sumeet DHAM, San Diego, CA (US); Randell L. WERNETH, San Diego, CA (US); Graydon Ernest BEATTY, San Diego, CA (US); Ben COPPOLA, Carlsbad, CA (US); Timothy J. CORVI, San Diego, CA (US)

(21) Appl. No.: 18/870,807

(22) PCT Filed: Jun. 6, 2023
(86) PCT No.: PCT/US2023/024547

§ 371 Date: Dec. 2, 2024

Related U.S. Application Data
(60) Provisional application No. 63/365,916, filed on Jun. 6, 2022.
(60) Provisional application No. 63/366,995, filed on Jun. 24, 2022.

(51) Int. Cl. A61N 1/39; A61N 1/05

(52) U.S. Cl. CPC A61N 1/395; A61N 1/0595; A61N 1/3956

(57) ABSTRACT

Systems, devices, and methods for providing post operative treatment of atrial fibrillation to a patient are described. The system includes: one or more leads, each lead having one or more electrodes for delivering energy to the heart of the patient; an energy delivery device for providing energy comprising a first waveform; and a converter device electrically connected to the energy delivery device. The converter device receives the energy comprising the first waveform from the energy delivery device, converts the energy having the first waveform into energy having a second waveform, and delivers the energy having the second waveform to the patient's heart via the one or more electrodes of each of the one or more leads. The energy having the second waveform delivered by the converter treats atrial fibrillation of the patient.

### RELATED APPLICATIONS

The present application claims priority to U.S. Provisional Patent Application Ser. No. 63/365,916, entitled “Post-Operative Devices and Therapy Methods Therefor”, filed Jun. 6, 2022, and to U.S. Provisional Patent Application Ser. No. 63/366,995, entitled “Post Operative Atrial Fibrillation”, filed Jun. 24, 2022, the contents of each of which is incorporated herein by reference in its entirety for all purposes.

The present application, while not claiming priority to, may be related to U.S. application Ser. No. 13/340,637, filed on Dec. 29, 2011, now U.S. Pat. 8,473,051 issued Jun. 25, 2013, which claims priority to U.S. Provisional Patent Application No. 61/428,098, filed on Dec. 29, 2010, each of which are incorporated by reference herein in their entireties.

The present application, while not claiming priority to, may be related to U.S. application Ser. No. 17/140,333, filed on Jan. 4, 2021, which is a continuation of U.S. application Ser. No. 13/947,840, filed on Jul. 22, 2013, now U.S. Pat. 10,905,884 issued Feb. 2, 2021, which claims priority to U.S. Provisional Patent Application No. 61/674,145, filed on Jul. 20, 2012, each of which are incorporated by reference herein in their entireties.

The present application, while not claiming priority to, may be related to U.S. application Ser. No. 18/038,526, filed on May 24, 2023, which is a 35 USC 371 national stage filing of International PCT Patent Application Serial Number PCT/US21/061257, filed on Nov. 30, 2021, which claims priority to U.S. Provisional Patent Application No. 63/119,970, filed on Dec. 1, 2020, and U.S. application Ser. No. 17/472,317, filed on Sep. 10, 2021, each of which are incorporated by reference herein in their entireties.

The present application, while not claiming priority to, may also be related to U.S. application Ser. No. 17/474,210, filed on Sep. 14, 2021, which is a continuation of U.S. application Ser. No. 17/472,317, filed on Sep. 10, 2021, which claimed priority to U.S. Provisional Patent Application No. 63/119,970, filed on Dec. 1, 2020, each of which is hereby incorporated by reference.

The present application, while not claiming priority to, may be related to International PCT Patent Application Serial Number PCT/US2022/036926, entitled “STIMULATION SYSTEM”, filed Jul. 13, 2022, which claims priority to U.S. Provisional Patent Application Ser. No. 63/336,517, entitled “STIMULATION SYSTEM”, filed Apr. 29, 2022 and U.S. Provisional Patent Application Ser. No. 63/326,190, entitled “STIMULATION SYSTEM”, filed Mar. 31, 2022, and U.S. Provisional Patent Application Ser. No. 63/221,117, entitled “STIMULATION SYSTEM”, filed Jul. 13, 2021, each of which is hereby incorporated by reference.

The present application, while not claiming priority to, may be related to U.S. application Ser. No. 17/637,877, entitled “Cardiac Stimulation System”, filed Feb. 24, 2022, which is a 35 USC 371 national stage filing of International PCT Patent Application Serial Number PCT/US2020/049349, entitled “Cardiac Stimulation System”, filed Sep. 4, 2020, Publication Number WO 2021/046313, published Mar. 11, 2021, which claimed priority to U.S. Provisional Application Ser. No. 62/895,655, entitled “Multi-Side Micro Pacing Circuits and Algorithms, Integrated into a Venously Placed Stent Assembly”, filed Sep. 4, 2019, each of which is hereby incorporated by reference.

The present application, while not claiming priority to, may be related to U.S. application Ser. No. 17/908,002, entitled “Cardiac Pacing Device”, filed Aug. 30, 2022, which is a 35 USC 371 national stage filing of International PCT Patent Application Serial Number PCT/US2021/021467, entitled “Cardiac Pacing Device”, filed Mar. 9, 2021, Publication Number WO 2021/183487, published Sep. 16, 2021 which claims priority to U.S. Provisional Patent Application Ser. No. 62/987,238, entitled “Stent, Mounted and Delivered Wireless, Batteryless Micropacing Chip System”, filed Mar. 9, 2020, each of which is hereby incorporated by reference.

The present application, while not claiming priority to, may be related to U.S. application Ser. No. 17/925,821, entitled “Pacing and Sensing Devices and Control System”, filed Nov. 16, 2022, which is a 35 USC 371 national stage filing of International PCT Patent Application Serial Number PCT/US2021/035132, entitled “Pacing and Sensing Devices and Control System”, filed Jun. 1, 2021, Publication Number WO2021/247490, published Dec. 9, 2021, which claims priority to U.S. Provisional Patent Application Ser. No. 63/032,687, entitled “Rechargeable Biomedical Battery Powered Wireless Self-Anchoring Micro-Pacing And Sensing Devices And Control System”, filed May 31, 2020, each of which is hereby incorporated by reference.

The present application, while not claiming priority to, may be related to U.S. Provisional Patent Application Ser. No. 63/389,094, filed Jul. 14, 2022, entitled “ADVANCED PACING”, which is hereby incorporated by reference.

The present application, while not claiming priority to, may be related to U.S. Provisional Patent Application Ser. No. 63/453,570, filed Mar. 21, 2023, entitled “ADVANCED PACING”, which is hereby incorporated by reference.

### FIELD OF THE INVENTIVE CONCEPTS

The present inventive concepts relate generally to stimulation systems, and in particular, systems that stimulate tissue of a patient's heart. BACKGROUND

The heart is a critical muscle in humans and many other animals that is responsible for circulating blood through the circulatory system. The human heart is made up of four chambers, two upper atria, and two lower ventricles, organized into a left and right pairing of an atrium and a ventricle. In a healthy heart, the chambers contract and relax in a synchronized fashion, referred to as a “beat,” in order to force blood through the network of veins and arteries.

Irregular heartbeats can pose a health risk, and in some cases normal beating can be restored via electrical stimulation. Implantable devices called “pacemakers” are devices which can stimulate the muscle tissue, causing it to contract. By methodically and accurately applying stimulation as needed, normal heart rhythm can be restored.

There is a need for improved systems for treating irregular heartbeats.

### SUMMARY

According to an aspect of the present inventive concepts, a system for providing post operative treatment of atrial fibrillation to a patient comprises: one or more leads, each lead comprising one or more electrodes for delivering energy to the heart of the patient; an energy delivery device for providing energy comprising a first waveform; and a converter device electrically connected to the energy delivery device. The converter device is configured to: receive the energy comprising the first waveform from the energy delivery device; convert the energy comprising the first waveform into energy comprising a second waveform; and deliver the energy comprising the second waveform to the patient's heart via the one or more electrodes of each of the one or more leads. The energy comprising the second waveform delivered by the converter treats atrial fibrillation of the patient.

In some embodiments, the second waveform comprises a multi-pulse therapy waveform.

The technology described herein, along with the attributes and attendant advantages thereof, will best be appreciated and understood in view of the following detailed description taken in conjunction with the accompanying drawings in which representative embodiments are described by way of example.

### INCORPORATION BY REFERENCE

All publications, patents, and patent applications mentioned in this specification are herein incorporated by reference to the same extent as if each individual publication, patent, or patent application was specifically and individually indicated to be incorporated by reference. The content of all publications, patents, and patent applications mentioned in this specification are herein incorporated by reference in their entirety for all purposes.

### BRIEF DESCRIPTION OF THE DRAWINGS

FIG. 1 illustrates a schematic view of a system for diagnosing and/or treating a patient, consistent with the present inventive concepts.

FIG. 1A illustrates a partial anatomic, partial schematic view of a system of devices, consistent with the present inventive concepts.

FIG. 2 illustrates a schematic view of a system for providing post operating treatment of atrial fibrillation, consistent with the present inventive concepts.

FIG. 3 illustrates a side view of a cardiac lead, consistent with the present inventive concepts.

FIG. 4 illustrates a side view of a cardiac lead and an anchoring clip, consistent with the present inventive concepts.

FIG. 5 illustrates a schematic view of a system of multiple devices collectively configured to convert a defibrillation waveform from a first device to a multi-pulse therapy waveform to be delivered to a second device, consistent with the present inventive concepts.

FIG. 6 illustrates a schematic view of a converter device operably attached to a limited functionality device, consistent with the present inventive concepts.

FIGS. 7A-F illustrate charts of various stimulation waveforms, consistent with the present inventive concepts.

FIG. 8 illustrates a schematic of a device configured to convert a defibrillation waveform from a first device to a multi-pulse therapy waveform to be delivered by a second device, consistent with the present inventive concepts.

FIG. 9 illustrates anatomic views of a heart showing various pacing configurations, consistent with the present inventive concepts.

FIG. 10 is a photograph of an electrode patch, consistent with the present inventive concepts.

FIGS. 11A-B illustrate a graph of biphasic and monophasic, respectively, multi-pulse therapy (MPT) waveforms, consistent with the present inventive concepts.

### DETAILED DESCRIPTION OF THE DRAWINGS

Reference will now be made in detail to the present embodiments of the technology, examples of which are illustrated in the accompanying drawings. Similar reference numbers may be used to refer to similar components. However, the description is not intended to limit the present disclosure to particular embodiments, and it should be construed as including various modifications, equivalents, and/or alternatives of the embodiments described herein.

It will be understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third, etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

It will be further understood that when a first element is referred to as being “in”, “on” and/or “within” a second element, the first element can be positioned: within an internal space of the second element, within a portion of the second element (e.g., within a wall of the second element); positioned on an external and/or internal surface of the second element; and combinations of one or more of these.

As used herein, the term “proximate”, when used to describe proximity of a first component or location to a second component or location, is to be taken to include one or more locations near to the second component or location, as well as locations in, on and/or within the second component or location. For example, a component positioned proximate an anatomical site (e.g., a target tissue location), shall include components positioned near to the anatomical site, as well as components positioned in, on and/or within the anatomical site.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g., rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence. Correspondingly, the terms “prevent”, “preventing”, and “prevention” shall include the acts of “reduce”, “reducing”, and “reduction”, respectively.

The term “and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

The term “one or more”, where used herein can mean one, two, three, four, five, six, seven, eight, nine, ten, or more, up to any number.

The terms “and combinations thereof” and “and combinations of these” can each be used herein after a list of items that are to be included singly or collectively. For example, a component, process, and/or other item selected from the group consisting of: A; B; C; and combinations thereof, shall include a set of one or more components that comprise: one, two, three or more of item A; one, two, three or more of item B; and/or one, two, three, or more of item C.

In this specification, unless explicitly stated otherwise, “and” can mean “or”, and “or” can mean “and”. For example, if a feature is described as having A, B, or C, the feature can have A, B, and C, or any combination of A, B, and C. Similarly, if a feature is described as having A, B, and C, the feature can have only one or two of A, B, or C.

As used herein, when a quantifiable parameter is described as having a value “between” a first value X and a second value Y, it shall include the parameter having a value of: at least X, no more than Y, and/or at least X and no more than Y. For example, a length of between 1 and 10 shall include a length of at least 1 (including values greater than 10), a length of less than 10 (including values less than 1), and/or values greater than 1 and less than 10.

The expression “configured (or set) to” used in the present disclosure may be used interchangeably with, for example, the expressions “suitable for”, “having the capacity to”, “designed to”, “adapted to”, “made to” and “capable of” according to a situation. The expression “configured (or set) to” does not mean only “specifically designed to” in hardware. Alternatively, in some situations, the expression “a device configured to” may mean that the device “can” operate together with another device or component.

As used herein, the term “threshold” refers to a maximum level, a minimum level, and/or range of values correlating to a desired or undesired state. In some embodiments, a system parameter is maintained above a minimum threshold, below a maximum threshold, within a threshold range of values, and/or outside a threshold range of values, such as to cause a desired effect (e.g., efficacious therapy) and/or to prevent or otherwise reduce (hereinafter “prevent”) an undesired event (e.g., a device and/or clinical adverse event). In some embodiments, a system parameter is maintained above a first threshold (e.g., above a first temperature threshold to cause a desired therapeutic effect to tissue) and below a second threshold (e.g., below a second temperature threshold to prevent undesired tissue damage). In some embodiments, a threshold value is determined to include a safety margin, such as to account for patient variability, system variability, tolerances, and the like. As used herein, “exceeding a threshold” relates to a parameter going above a maximum threshold, below a minimum threshold, within a range of threshold values and/or outside of a range of threshold values.

As described herein, “room pressure” shall mean pressure of the environment surrounding the systems and devices of the present inventive concepts. Positive pressure includes pressure above room pressure or simply a pressure that is greater than another pressure, such as a positive differential pressure across a fluid pathway component such as a valve. Negative pressure includes pressure below room pressure or a pressure that is less than another pressure, such as a negative differential pressure across a fluid component pathway such as a valve. Negative pressure can include a vacuum but does not imply a pressure below a vacuum. As used herein, the term “vacuum” can be used to refer to a full or partial vacuum, or any negative pressure as described hereabove.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross sectional area as the cross section of the component being described.

The terms “major axis” and “minor axis” of a component where used herein are the length and diameter, respectively, of the smallest volume hypothetical cylinder which can completely surround the component.

As used herein, the term “functional element” is to be taken to include one or more elements constructed and arranged to perform a function. A functional element can comprise a sensor and/or a transducer. In some embodiments, a functional element is configured to deliver energy and/or otherwise perform a treatment on tissue (e.g., a functional element configured as a treatment element). Alternatively or additionally, a functional element (e.g., a functional element comprising a sensor) can be configured to record one or more parameters, such as a patient physiologic parameter; a patient anatomical parameter (e.g., a tissue geometry parameter); a patient environment parameter; and/or a system parameter. In some embodiments, a sensor or other functional element is configured to perform a diagnostic function (e.g., to gather data used to perform a diagnosis). In some embodiments, a functional element is configured to perform a therapeutic function (e.g., to deliver therapeutic energy and/or a therapeutic agent). In some embodiments, a functional element comprises one or more elements constructed and arranged to perform a function selected from the group consisting of: deliver energy; extract energy (e.g., to cool a component); deliver a drug or other agent; manipulate a system component or patient tissue; record or otherwise sense a parameter such as a patient physiologic parameter or a system parameter; and combinations of one or more of these. A functional element can comprise a fluid and/or a fluid delivery system. A functional element can comprise a reservoir, such as an expandable balloon or other fluid-maintaining reservoir. A “functional assembly” can comprise an assembly constructed and arranged to perform a function, such as a diagnostic and/or therapeutic function. A functional assembly can comprise an expandable assembly. A functional assembly can comprise one or more functional elements.

The term “transducer” where used herein is to be taken to include any component or combination of components that receives energy or any input, and produces an output. For example, a transducer can include an electrode that receives electrical energy, and distributes the electrical energy to tissue (e.g., based on the size of the electrode). In some configurations, a transducer converts an electrical signal into any output, such as: light (e.g., a transducer comprising a light emitting diode or light bulb), sound (e.g., a transducer comprising a piezo crystal configured to deliver ultrasound energy); pressure (e.g., an applied pressure or force); heat energy; cryogenic energy; chemical energy; mechanical energy (e.g., a transducer comprising a motor or a solenoid); magnetic energy; and/or a different electrical signal (e.g., different than the input signal to the transducer). Alternatively or additionally, a transducer can convert a physical quantity (e.g., variations in a physical quantity) into an electrical signal. A transducer can include any component that delivers energy and/or an agent to tissue, such as a transducer configured to deliver one or more of: electrical energy to tissue (e.g., a transducer comprising one or more electrodes); light energy to tissue (e.g., a transducer comprising a laser, light emitting diode and/or optical component such as a lens or prism); mechanical energy to tissue (e.g., a transducer comprising a tissue manipulating element); sound energy to tissue (e.g., a transducer comprising a piezo crystal); chemical energy; electromagnetic energy; magnetic energy; and combinations of one or more of these.

As used herein, the term “fluid” can refer to a liquid, gas, gel, or any flowable material, such as a material which can be propelled through a lumen and/or opening.

As used herein, the term “material” can refer to a single material, or a combination of two, three, four, or more materials.

It is appreciated that certain features of the inventive concepts, which are, for clarity, described in the context of separate embodiments, may also be provided in combination in a single embodiment. Conversely, various features of the inventive concepts which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the inventive concepts have been simplified to focus on elements that are relevant for a clear understanding of the inventive concepts, while eliminating, for purposes of clarity, other elements that those of ordinary skill in the art will appreciate may also comprise a portion of the inventive concepts. However, because such elements are well known in the art, and because they do not necessarily facilitate a better understanding of the inventive concepts, a description of such elements is not provided herein.

Terms defined in the present disclosure are only used for describing specific embodiments of the present disclosure and are not intended to limit the scope of the present disclosure. Terms provided in singular forms are intended to include plural forms as well, unless the context clearly indicates otherwise. All of the terms used herein, including technical or scientific terms, have the same meanings as those generally understood by an ordinary person skilled in the related art, unless otherwise defined herein. Terms defined in a generally used dictionary should be interpreted as having meanings that are the same as or similar to the contextual meanings of the relevant technology and should not be interpreted as having ideal or exaggerated meanings, unless expressly so defined herein. In some cases, terms defined in the present disclosure should not be interpreted to exclude the embodiments of the present disclosure.

Provided herein are systems, devices, and methods for providing therapy to a heart of a patient. The system can comprise one or more implantable devices, such as one or more devices that are placed within the patient for a limited period of time (e.g., a temporary implant, such as a device that remains implanted for less than one month or less than one week) and/or remains implanted for an extended period of time (e.g., a chronically placed device, such as a device that remains implanted for at least one month, or at least six months). In some embodiments, the system includes one or more external devices, such as external devices that deliver power and/or data to one or more implantable devices. In some embodiments, a device includes both an implantable portion and an external portion, such as a device including a lead that extends from a location outside the patient's body, through the skin, to a location within the patient's body. An implantable device can comprise: an anchor configured to temporarily or chronically maintain the position of the implantable device; at least one sensor configured to record electrical activity of the heart; and/or one, two, or more electrodes and/or coils (“electrodes” herein) configured to deliver stimulation energy to tissue of the heart, such as to treat an arrhythmia such as atrial fibrillation (AF). The one, two, or more electrodes can be included on one, two, or more leads. The system can include a controller that comprises one or more algorithms, such as an algorithm that initiates and/or adjusts delivery of energy to treat the patient (e.g., to treat an arrhythmia of the patient). The system can include a first device that produces a first form of stimulation energy, and a second device that converts the first form of stimulation energy to a second form of stimulation energy (e.g., a multi-pulse therapy waveform as described herein). The second form of stimulation energy can comprise one, two, or more energy parameters that are different than those of the first form of stimulation energy, such as a parameter selected from the group consisting of: amplitude; frequency; pulse width; duration; amount of energy (e.g., total amount of energy delivered); quantity of pulses; pulse type (e.g., monophasic or biphasic); timing of pulses; synchronization delay times; and combinations of these.

Postoperative atrial fibrillation (POAF) is a common occurrence among patients who have had a cardiac surgical procedure (e.g., about 40% of cardiac surgical procedures are complicated by atrial fibrillation). Adverse consequences of POAF may include hemodynamic instability, increased risk of stroke, greater costs of healthcare, and lengthened hospital and intensive care unit (ICU) stays (e.g., an additional two to four days of hospital stay). For some patients, POAF may subside relatively quickly on its own, in which case no further treatment may be required. However, some patients may need additional treatment to resolve one or more undesired medical conditions.

Although a variety of treatments currently exist to address POAF, these conventional treatments are sub-optimal. In one treatment, patients experiencing AF for more than a few hours may be placed on an anticoagulant, such as Coumadin. Such a drug, however, may not be effective in all cases and may produce harmful and/or unwanted side effects. In another treatment, patients may receive a cardioversion procedure in which shocks (e.g., short-duration electrical shocks, such as short-duration high-energy electrical shocks) are utilized to restore a regular heart rhythm. This procedure, however, may impose undesirably delayed treatment and additional financial costs. Accordingly, a need exists for a cost-effective method of more effectively treating patients experiencing POAF than the conventional treatments described above.

Also provided herein are systems, methods, and devices for identifying AF (particularly POAF) events and delivering effective treatments. The system can include one or more electrodes (e.g., one or more leads, each lead containing one or more electrodes) that are configured to be placed onto various positions of a patient's heart (e.g., positions that avoid suboptimal cardiac signal recording and/or suboptimal energy delivery, such as positions that avoid epicardial fat pads). An implantable device connected to the leads can be configured to receive energy from another device (e.g., an external device such as an external defibrillator or an external pacer), to convert that energy to a multi-pulse therapy (MPT), such as to deliver an MPT signal (also referred to as an “MPT waveform”) to the heart via the leads.

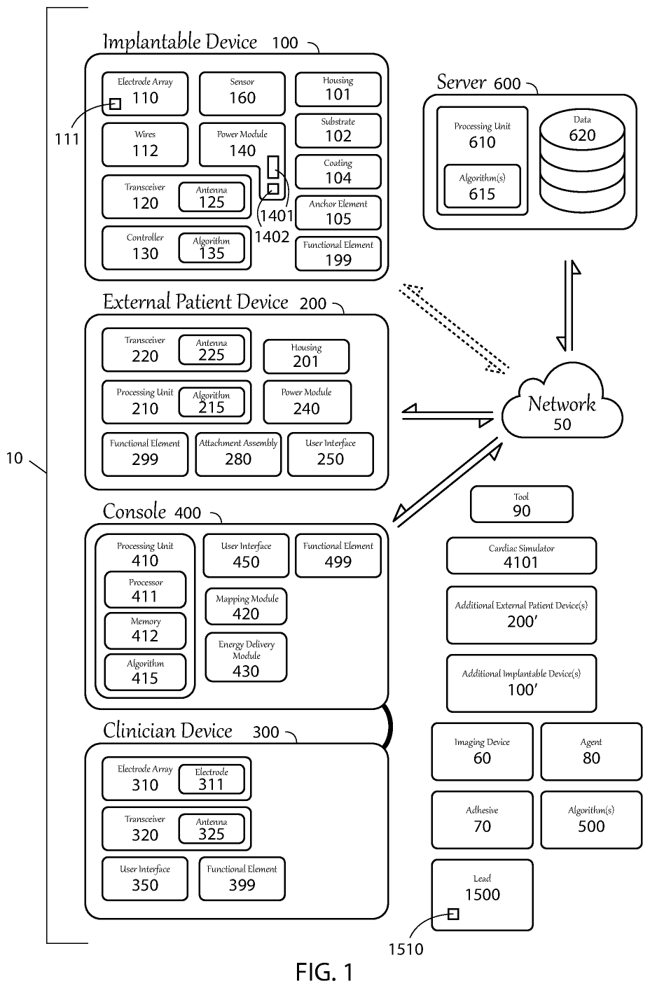

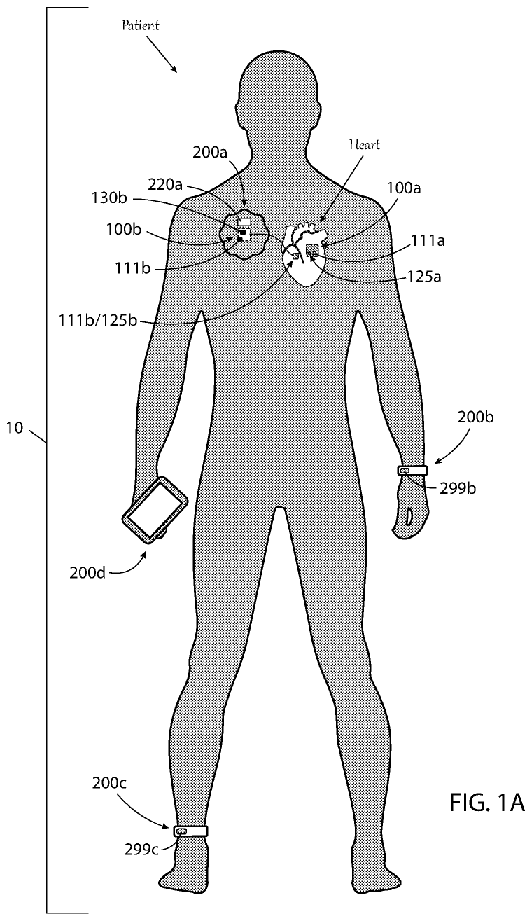

Referring now to FIG. 1, a schematic view of a system for diagnosing and/or treating a patient is illustrated, consistent with the present inventive concepts. System 10 can comprise one or more devices (e.g., devices for a clinician to perform a procedure, devices for a patient to position proximate their body, and/or devices for implantation in the patient) which can be configured to monitor one or more patient parameters, diagnose one or more patient conditions, and/or to treat one or more patient conditions, such as to treat a condition based on one or more patient diagnoses determined by system 10. For example, system 10 can be configured to monitor, diagnose, and/or treat (“treat” herein) an arrhythmia such as atrial fibrillation (AF), a category of abnormally fast and/or “highly irregular” rhythm due to improper electrical activity in the atrial chambers of the heart, such as by monitoring the electrical activity of the patient's heart, and by pacing the muscular tissue in either or both the atrial chambers of the heart to restore sinus rhythm when fibrillation is detected. In another example, system 10 can be configured to treat supraventricular tachycardia (SVT), a category of abnormally fast and/or “regular or quasi-regular” rhythms due to improper electrical activity in the atrial chambers of the heart, such as by monitoring the electrical activity of the patient's heart, and by pacing the muscular tissue in either or both the atrial chambers of the heart to restore sinus rhythm when SVT is detected. In another example, system 10 can be configured to treat atrial tachycardia (AT), a common abnormally fast and regular arrythmia in the category of SVT due to improper electrical activity in the atrial chambers of the heart, such as by monitoring the electrical activity of the patient's heart, and by pacing the muscular tissue in either or both the atrial chambers of the heart to restore sinus rhythm when AT is detected. In another example, system 10 can be configured to treat both the typical and atypical forms of atrial flutter (AFL), which are common abnormally fast and regular arrythmias in the category of SVT due to improper electrical activity in the atrial chambers of the heart, such as by monitoring the electrical activity of the patient's heart, and by pacing the muscular tissue in either or both the atrial chambers of the heart to restore sinus rhythm when AFL is detected. System 10 can include one or more devices configured to be implanted, implantable device 100 (also referred to as ID 100 herein), which can be implanted into the patient for an extended period of time (e.g., at least 1 month, at least 3 months, and/or at least 6 months), such as when implanted by a clinician during a clinical procedure. In some embodiments, implantable device 100 comprises a short-term implant, such as when implantable device 100 is configured to be implanted for no more than 6 months, no more than 3 months, and/or no more than 1 month (e.g., implantable device 100 can be implanted for two or three weeks following cardiac surgery). In some embodiments, ID 100 is configured to be implanted during a cardiac surgical procedure (e.g., an open chest procedure), and ID 100 is configured to be placed on the epicardial surface and adhered thereto, such as with suture, compression, and/or surgical glue.

In some embodiments, system 10 comprises one or more externally-placed devices, external patient device 200, which can comprise one or more devices that are configured to monitor, diagnose, and/or treat a patient, such as from one or more locations outside the patient's body. Alternatively or additionally, external patient device 200 (also referred to as EPD 200) can be configured to communicate (e.g., wirelessly communicate) with implantable device 100 (also referred to as ID 100), such as to transfer data between EPD 200 and ID 100, and/or to transfer power from EPD 200 to ID 100. In some embodiments, EPD 200 is configured as a stimulator, such as a cardiac stimulator. For example, EPD 200 can be configured as a pacemaker, a cardioverter, a defibrillator, and/or any combination of two or three of these. In some embodiments, ID 100 comprises two devices, a first device configured to be implanted proximate the patient's heart, as described herein, and a second device configured to be implanted at another location under the patient's skin (e.g., subcutaneously). In some embodiments, the second ID 100 (implanted subcutaneously) is configured similar to EPD 200 described herein, such as to transmit power and/or data to the first ID 100 (implanted proximate the patient's heart). In these embodiments, system 10 may include, or may not include, EPD 200. Alternatively or additionally, system 10 may include at least two IDs 100 (such as ID 100a and 100b shown in FIG. 1A), where a first ID 100 is configured as EPD 200 and to transmit power and/or data to a second ID 100, and also include an EPD 200, such as when EPD 200 is configured to transmit power to the first ID 100, for example, to recharge a power supply (e.g., a battery and/or a capacitor) of the first ID 100. In some embodiments, one or more IDs 100 and/or EPDs 200 can be operably connected via one or more conduits, not shown, but such as an electrical conduit that is tunneled beneath the skin of the patient (e.g., to connect a subcutaneously implanted ID 100 and/or EPD 200 to an ID 100 implanted proximate the heart).

In some embodiments, ID 100 can comprise one or more housings, housing 101 shown, that surrounds one or more components of ID 100, such as one or more computing, signal generating, and/or power handling components of ID 100, such as are described herein. In some embodiments, EPD 200 can comprise one or more housings, housing 201 shown, that surrounds one or more components of EPD 200, such as one or more computing, signal generating, and/or power handing components of EPD 200, such as are described herein. In some embodiments, one or more of electrodes (e.g., electrodes 111 shown) and/or other functional elements (e.g., functional element 199) of ID 100, and/or one or more functional elements (e.g., functional element 299) of EPD 200, are located outside of housings 101 and/or 201, respectively, such as when one or more functional elements are located on the outside of the housing of the respective devices, and/or are operably connected to the respective devices, such as via a lead or other electrical conduit as described herein.

System 10 can be configured to monitor for and/or to detect irregular or otherwise undesirable (“irregular” or “undesirable” herein) electrical conduction signals or patterns (“patterns” herein) in tissue and/or to deliver energy to the tissue to restore a desirable regular (e.g., healthy) electrical conduction pattern. In some embodiments, system 10 is configured to detect undesirable conduction patterns comprising regular but rapid patterns. System 10 can be configured to monitor the electrical activity of the heart (e.g., conduction patterns proximate the left and/or right atrium of the heart), and to detect the presence of irregular conduction patterns, such as conduction patterns indicative of AF and/or SVT. Additionally or alternatively, system 10 can deliver electrical energy (e.g., pacing pulses) to tissue exhibiting irregular conduction patterns, as well as to tissue surrounding that tissue, to alter the irregular conduction patterns. In some embodiments, system 10 is configured to deliver “multi-site” pacing, where pacing energy is delivered from two, three, four, or more electrodes positioned at different locations, such as different locations proximate the left atrium and/or the right atrium. For example, system 10 can be configured to deliver multi-site left-atrial pacing (i.e., delivery of energy to two left atrial tissue locations) configured to restore sinus rhythm in patients exhibiting irregular conduction patterns. In some embodiments, system 10 is configured to ablate tissue, such as by delivering energy configured to thermally ablate and/or irreversibly electroporate tissue. In these embodiments, system 10 can be further configured to also deliver pacing energy to tissue, such as multi-site pacing energy and/or other pacing energy, such as is described herein.

In some embodiments, system 10 is configured to deliver energy to the patient's heart (e.g., to treat a regular and/or an irregular arrhythmia such as atrial fibrillation, and/or other irregular heartbeat) at a level such that the energy delivery is not perceived by the patient, or at least minimally perceived by the patient (e.g., at a level below a pain threshold). In some embodiments, this energy delivery comprises multiple site energy delivery as described herein (e.g., spatially distanced energy delivery). For example, multiple electrodes (e.g., at least three, four, or five electrodes) can be placed on the epicardium for delivery of the stimulation energy. These multiple electrodes can be spatially distributed to gain sufficient coverage of the heart chamber to achieve effective therapy, such as to effectively terminate (e.g., pace terminate) atrial fibrillation and/or other arrhythmia. In some embodiments, less than 6 mJ (e.g., less than 5 mJ or less than 4 mJ) of energy can be delivered at each epicardial site (e.g., over a period of approximately one second), such as to terminate atrial fibrillation in the patient.

System 10 can include one or more devices for use by a clinician during a clinical procedure, clinician device 300. Clinician device 300 (also referred to as CD 300) can comprise one or more delivery devices, such as a kit of devices configured to enable the clinician to perform an implantation procedure for implanting ID 100 into the patient. For example, CD 300 can comprise one or more delivery catheters, such as when ID 100 is configured to be implanted during a minimally invasive procedure, such as an interventional procedure performed in a catheterization laboratory (often referred to as a “cath lab”). For example, CD 300 can comprise one or more tools for percutaneous delivery of ID 100 in the patient's vasculature, and one or more tools for transvascular delivery of ID 100 into locations outside of the patient's vasculature (e.g., into the pericardial space, such as onto the epicardial surface). Alternatively or additionally, CD 300 can comprise one or more surgical tools (e.g., minimally invasive tools) for surgically implanting ID 100 (e.g., a surgical access kit for use in an operating room). For example, CD 300 can comprise one or more surgical tools for percutaneous delivery of ID 100 (e.g., a needle-based tool for insertion of ID 100 into the pericardial space). In some embodiments, ID 100 comprises a first geometry where ID 100 is in an undeployed state, such as a geometry comprising a collapsed, folded, or otherwise undeployed geometry configured to allow ease of insertion into the patient. ID 100 can be configured to transition from the first geometry into a second geometry in which ID 100 is in an expanded or otherwise deployed state. In some embodiments, ID 100 is configured to be deployed from a coronary vessel (e.g., through the tissue wall) and implanted along the epicardial surface (e.g., during an interventional procedure), such as is described in detail herein. For example, ID 100 can be deployed from a vessel (e.g., a coronary vessel) selected from the group consisting of: the coronary sinus; the Great Cardiac Vein; the Vein of Marshall; the Azygos vein; a side-branch that anastomoses the coronary sinus, for example, side branches that lie proximate a desired deployment location such as the epicardial surface of the left atrium; and combinations of these. In some embodiments, ID 100 can be configured to be deployed by a robotic delivery device, such as a magnetically-driven robotic device.

In some embodiments, CD 300 includes one or more tools for providing epicardial access (e.g., subxiphoid percutaneous epicardial access), such as to allow a clinician to implant ID 100 on or otherwise proximate an epicardial surface. CD 300 can be configured to prevent (or at least limit the likelihood) of ventricular puncture. CD 300 can be constructed and arranged to enable the clinician to perform a “dry tap” of the epicardial space (e.g., without allowing the needle to penetrate the ventricular tissue). In some embodiments, CD 300 includes one or more devices for positioning a visualizable device, such as a visualizable portion of guidewire or a lead (e.g., a visualizable lead, such as a lead visualizable under fluoroscopy or ultrasound) proximate the lateral margin of the roof of the right atrium (RA). In some embodiments, CD 300 includes a needle and mechanical or other stopping mechanism configured to prevent the needle from advancing into ventricular tissue. After placement, this visualizable device can assist the clinician by providing a visualizable marker indicating the location of the lateral RA boundary. In some embodiments, CD 300 includes one or more devices for positioning a visualizable device proximate the posterior wall of the left atrium (LA). After placement, this visualizable device can assist the clinician by providing a visualizable marker indicating the location of the posterior LA boundary. In some embodiments, CD 300 includes one or more devices for positioning a visualizable device proximate the interatrial septum between the RA and LA. After placement, this visualizable device can assist the clinician by providing a visualizable marker indicating the location of the interatrial septum. In some embodiments, CD 300 includes one or more devices for positioning a visualizable device proximate the apex of the right ventricle (RV). After placement, this visualizable device can assist the clinician by providing a visualizable marker indicating the location of the RV boundary. In some embodiments, CD 300 includes one or more devices for positioning a lead (e.g., a visualizable lead, such as a lead visualizable under fluoroscopy or ultrasound) proximate the apex of the left ventricle (LV). After placement, this lead can assist the clinician by providing a visualizable marker indicating the location of the LV boundary. In some embodiments, system 10 is configured to image the RV, such as with an angiogram or other visualization method (e.g., as provided by imaging device 60 described herein), such as to assist the clinician by providing one or more images that show the border of the RV and the pericardial space. In some embodiments, system 10 is configured to image the LV, such as with an angiogram or other visualization method (e.g., as provided by imaging device 60 described herein), such as to assist the clinician by providing one or more images that show the border of the LV and the pericardial space.

In some embodiments, CD 300 includes a device (e.g., a needle) configured to provide a signal used to identify the pericardial juncture (e.g., by providing a bioimpedance signal). For example, the needle can include an electrode proximate the distal end of the needle. Alternatively or additionally, the needle can comprise an electrically conductive material, and at least a proximal portion of the needle can be insulated such that the distal tip of the needle comprises the electrode. In some embodiments, CD 300 comprises one or more devices configured to be positioned within the coronary sinus, perforate the coronary sinus, and enter the pericardial space. System 10 can comprise one or more visualizable agents, agent 80 shown. In some embodiments, CD 300 is constructed and arranged to inject agent 80 (e.g., a radiopaque material such as contrast) into the pericardial space. For example, device 300 can be configured to inject approximately 10 mL of a contrast-based agent 80 into the pericardial space.

In some embodiments, clinician device 300 comprises a programmer configured to transfer a set of parameters (e.g., a “program”) to one or more of implantable device 100 and/or external patient device 200. In some embodiments, external patient device 200 can transfer programs to implantable device 100. A program can comprise a set of parameters, such as stimulation parameters which implantable device 100 will follow when stimulating the patient, such as described herein. In some embodiments, algorithm 135 is configured to cause implantable device 100 to stimulate the patient based on a program received from EPD 200 and/or clinician device 300.

System 10 can include one or more consoles, console 400 shown. Console 400 can operably connect to CD 300 and can be configured to facilitate one or more processes, energy deliveries, data collections, data analyses, data transfers, signal processing, and/or other functions (“functions” herein) of system 10. In some embodiments, system 10 is constructed and arranged to map electrical activity within the body of a patient (e.g., to map electrical activity of the patient's heart), such as when CD 300 comprises a mapping catheter and console 400 comprises mapping module 420. Mapping module 420 can be configured to record and process mapping signals recorded by CD 300. For example, mapping module 420 can be configured to characterize conduction patterns and/or signal morphologies, such as to classify them (e.g., via algorithm 415 described herein) into arrythmia types, such as AF, AT, AFL, and the like, both typical and atypical. In some embodiments, implantable device 100 and/or EPD 200 are similarly configured to characterize conduction patterns and/or signal morphologies, for example, by processing signals recorded by electrode array 110 (e.g., processing via algorithms 135 and/or 215). In some embodiments, system 10 is constructed and arranged to ablate tissue (e.g., ablate cardiac tissue to treat AF). In these embodiments, console 400 comprises energy delivery module 430. Energy delivery module 430 can be configured to deliver ablative energy to tissue, such as via one or more energy delivery elements (e.g., electrodes, ultrasound transducers, light-emitting elements, and the like) of CD 300. In some embodiments, system 10 is constructed and arranged to stimulate tissue, for example, by delivering stimulation energy via one or more electrodes 311 and/or other energy delivery elements of clinician device 300 (e.g., as described herein). Energy delivery module 430 can be configured to deliver energy in the form of stimulation pulses that stimulate tissue. Energy delivery module 430 can deliver stimulation pulses via any one or more single electrodes 311, and/or via one or more sets (e.g., pairs) of electrodes 311 at any given instant and/or at any frequency. In some embodiments, stimulation pulses are delivered as a sequence of pulses, such as a sequence of pulses that are delivered simultaneously and/or asynchronously, and/or regularly and/or irregularly. The stimulation pulses can be delivered across a plurality of operably connected electrodes 311, where each electrode 311 can be positioned at prescribed (e.g., clinician and/or system 10 determined, as described herein) locations about a chamber and/or chambers of the heart. These sequences of pulses can be controlled either manually and/or by an algorithm (e.g., algorithm 415 described herein), such as an algorithm that determines the location and the instances in time to deliver stimulation, such as a determination that is based on the measured state of a prescribed chamber's conduction pattern. Console 400 can include processing unit 410, which can be configured to perform one or more functions of console 400 (e.g., as described hereabove). Processing unit 410 can include processor 411, memory 412, and/or algorithm 415, each as shown. In some embodiments, memory 412 stores instructions to perform algorithm 415. Processing unit 410 can be constructed and arranged to execute algorithm 415 and to thereby execute one or more functions of console 400. In some embodiments, console 400 includes one or more user interfaces, user interface 450. In some embodiments, console 400 includes one or more functional elements, functional element 499 shown. Functional element 499 can include one or more sensors and/or transducers.

In some embodiments, console 400 is configured to perform a diagnostic interrogation of the morphology of cardiac activity of the patient, such as to provide a diagnostic interrogation of AF and/or SVT. For example, algorithm 415 can analyze electrical activity of the patient's heart to determine a treatment plan including selection and configuration of one or more components of system 10 to optimize treatment of the patient. In some embodiments, algorithm 415 is configured to process one or more electrograms (e.g., electrograms recorded by system 10 and/or imported into system 10) to produce a 3D model of the electrical activity of at least a portion of the heart. For example, algorithm 415 can produce 3D models that can be displayed (e.g., via user interface 450) to show the electrical conduction patterns and/or conduction timing of a portion of the heart (e.g., one or more chambers of the heart).

The implantation locations of each of the electrodes 111 can be: determined automatically by system 10, determined by a clinician, and/or determined in a semi-automated way based on clinician and system 10 input. In some embodiments, a pacing diagnostic procedure is performed in which energy is delivered by an electrode (e.g., an electrode 311 of clinician device 300, an electrode 111 of ID 100, and/or other electrode) that is positioned in a tissue location temporarily, such as to assess the impact (the pacing impact) of the energy delivery at that location (e.g., an epicardial or other cardiac location). In these embodiments, a set of electrodes 111 implant locations can be determined. In some embodiments, additional criteria can be used to determine the electrode 111 implant locations, such as clinical criteria, anatomical criteria, geometric criteria, criteria derived in simulations, and/or other criteria. These additional criteria can be used cooperatively with the criteria collected in the pacing diagnostic procedure to determine the implant locations for the electrodes 111.

System 10 can include one or more imaging devices, imaging device 60. Imaging device 60 can comprise an imaging device selected from the group consisting of: an X-ray device such as a fluoroscopy device; a CT scanner device; an MRI device; an ultrasound imaging device; and combinations of these.

ID 100 can comprise one or more arrays of functional elements (e.g., sensors and/or transducers), electrode array 110, comprising one, two or more elements, electrodes 111. Electrode array 110 can comprise multiple configurations. Electrode array 110 can be constructed and arranged to be implanted on the epicardial surface of the heart, for example, on the epicardial wall proximate the left atrium. Alternatively or additionally, at least a portion of electrode array 110 (e.g., at least one electrode 111) can be implanted in a vessel (e.g., a coronary vessel), such as the coronary sinus, the Vein of Marshall, the Azygos vein, and/or another vessel proximate the heart. In some embodiments, at least a portion of electrode array 110 is implanted on the endocardial surface, such as within the left atrium or right atrium of the heart, for example, on the septum between the left and right atrium. In some embodiments, at least a portion of electrode array 110 is positioned within the left atrial appendage (e.g., as part of a left atrial appendage closure device). Electrodes 111 can comprise pacing electrodes configured to deliver electrical stimulation energy to patient tissue (e.g., tissue of the heart). Additionally or alternatively, electrodes 111 can comprise sensing electrodes configured to record electrical activity of tissue (e.g., electrical activity of the heart). Electrodes 111 can be configured to deliver electrical stimulation and/or to sense electrical activity in unipolar and/or multipolar (e.g., bipolar) configurations, such as when two electrodes 111 comprise a pair of electrodes configured to operate in a source and sink arrangement. In some embodiments, electrode array 110 is fixedly attached to one or more flexible membranes, substrate 102. In some embodiments, substrate 102 comprise a single layer membrane. In some embodiments, substrate 102 comprises two or more membrane layers. Substrate 102 can comprise an elastomeric material, for example, a material selected from the group consisting of: poly(lactic-co-glycolic) acid (PLGA); silicone (PDMS); liquid crystal polymers; polyimide; polyurethane (PU); thermoplastic polyurethane (TPU); and combinations of these. In some embodiments, substrate 102 comprises a fabric mesh, such as a polyester mesh. In some embodiments, ID 100 can comprise an anchoring element, such as anchoring element 105 described herein. In some embodiments, substrate 102 comprises a flexible material. In some embodiments, substrate 102 comprises a stretchable material, for example, a material that can stretch at least 5% and/or a material that stretches no more than 200%. In some embodiments, substrate 102 can comprise one or more holes constructed and arranged to cause and/or enhance capillary action of tissue into substrate 102.

In some embodiments, one or more components of ID 100 (e.g., the components on the outer surfaces of ID 100 which will be exposed to the environment within the body when implanted) comprise biocompatible materials. In some embodiments, one or more components of ID 100 are at least partially encapsulated within substrate 102, for example, electrode array 110 can be positioned between two or more layers of substrate 102. In some embodiments, at least a portion of each electrode 111 of electrode array 110 extends through a layer of substrate 102 (e.g., an outer layer) such that at least a surface portion of electrode 111 is exposed to the body when ID 100 is implanted (e.g., such that ID 100 can be positioned with one or more electrodes 111 (e.g., all electrodes 111) in contact with the epicardial wall). In some embodiments, electrode array 110, and/or other electronic components of ID 100 can comprise one or more elastomeric materials. Alternatively or additionally, electrode array 110, and/or other electronic components of ID 100 can comprise one or more non-elastomeric materials. In some embodiments, substrate 102 comprises an elongate tubular geometry, such as when substrate 102 comprises a shaft similar to a catheter shaft with one or more electrodes positioned thereon.

In some embodiments, electrodes 111 comprise a coating and/or a surface treatment (either or both, “coating” herein), such as a coating that is configured to enhance the recording ability of ID 100 via electrodes 111. For example, electrode 11 can comprise one or more coatings that are configured to increase the surface area of electrodes 111, such as to enhance the recording ability of electrodes 111, such as by lowering the source impedance of electrodes 111. Coatings can reduce the impedance and effects of the half-cell potential that occur from large surface areas of noble metals. A balance of low input impedance and reduced capacitive effects are needed here. Consider that small electrodes are noisy due to high source impedance.

Electrode array 110 can comprise an array surface area (e.g., the surface area defined by the outside boundary of array 110, and/or the convex hull of electrodes 111 of ID 100) of at least 6.5 cm². In some embodiments, electrode array 110 can comprise a surface area greater than or equal to at least 12% of the epicardial surface of the right atrium of the heart and/or the left atrium of the heart. System 10 can comprise multiple implantable devices 100 in various sizes and shapes (e.g., various array 110 sizes), such as when provided in a kit form such that a clinician can select which implantable device 100 of a kit of implantable devices 100 to implant. The selection made can be based on one or more patient parameters, such as the size of the patient's heart (e.g., the size of an atrium and/or a ventricle of the patient's heart). In some embodiments, the size of array 110 of a particular ID 100 is proportional to the amount of tissue through which ID 100 can manipulate the electrical activity of the heart (e.g., to control and/or direct the propagation of cardiac activation of the tissue). In some embodiments, one or more ID 100 can be implanted at a location selected to treat a particular disease or ailment. For example, ID 100 can be implanted proximate the left atrium (e.g., on the epicardial surface) to deliver stimulation energy to treat atrial fibrillation. Alternatively or additionally, ID 100 can be implanted proximate a ventricle of the heart (e.g., on the epicardial surface) to deliver stimulation energy to treat ventricular tachycardia and/or ventricular fibrillation. In some embodiments, at least one electrode 111 is implanted in each chamber of the heart (e.g., at least one ID 100 is implanted in each chamber of the heart), such that system 10 can sense and/or pace from within each chamber.

In some embodiments, ID 100 comprises multiple devices, such as at least 5, or at least 10 devices. In these embodiments, multiple implantable devices 100 can be configured to be implanted in a distributed manner, for example, evenly distributed across one or more portions of the epicardial surface. In some embodiments, multiple devices 100 are configured to treat the patient in a coordinated fashion, such as to deliver energy to the cardiac tissue in a pattern based on the location of each individual ID 100 (e.g., relative to each other and/or the cardiac tissue). In some embodiments, ID 100 can comprise multiple devices. In some embodiments, multiple devices 100 are configured to collectively treat a patient with multiple arrhythmias in a coordinated fashion, such as to deliver energy to the right and/or the left atrium to treat AF and/or SVT, and/or to deliver energy to the right and/or the left ventricle to treat other arrhythmias. For example, the multiple devices 100 can each deliver energy as needed (e.g., as determined by a treatment plan of system 10, described herein), such as when the device 100 closest to the source of an arrhythmia is selected to deliver energy to treat that arrhythmia. In some embodiments, at least one of a set of multiple implantable devices 100 can be configured to be implanted in one or more locations selected from the group consisting of: within the right atrium, such as affixed to the endocardial wall of the right atrium; within the left atrium, such as affixed to the endocardial wall of the left atrium; within the left and/or right ventricle; proximate one or more pulmonary veins, such as within and/or partially surrounding a pulmonary vein; on the endocardial surface proximate the left and/or right atrium, the left and/or right ventricle, a pulmonary vein, and/or another anatomic landmark; within a coronary vessel; embedded into cardiac tissue, such as between the endocardial and epicardial surface; and combinations of these.

In some embodiments, one or more conductive portions (e.g., conductive surfaces) of ID 100 (e.g., conductive portions of electrode array 110) are positioned on device 100 to be directed towards tissue to be stimulated when ID 100 is implanted (e.g., directed towards cardiac tissue), and one or more nonconductive portions of ID 100 are positioned on device 100 to be directed toward tissue to be insulated from stimulation energy delivered by ID 100 (e.g., directed toward the pericardium). For example, ID 100 can be configured to be implanted on the epicardial surface with the “bottom” of ID 100 directed towards the epicardial surface, and electrodes 111 can be positioned on the bottom of ID 100 and insulated from the top of ID 100, such as to prevent unintended stimulation of the phrenic nerve, the pericardium, and/or other electrically active thoracic structures. In some embodiments, ID 100 can comprise a cover configured to insulate one or more portions of ID 100 from tissue.

In some embodiments, system 10 includes one or more electrical conduits, lead 1500 shown, which can be configured to operably attach two or more components of system 10, such as two devices of system 10, and/or a device of system 10 and one or more functional elements, for example, one or more electrodes located on lead 1500. For example, lead 1500 can comprise one or more functional elements (e.g., sensors and/or transducers), such as electrode 1510 shown, which can be positioned on a distal portion of lead 1500 (e.g., when the distal portion of lead 1500 is configured to be implanted proximate cardiac tissue to be diagnosed and/or otherwise treated by system 10 via electrodes 1510). Lead 1500 can be tunneled or otherwise routed (e.g., in a surgical or minimally invasive procedure) between the cardiac tissue and an implant location of housing 101 of ID 100. Alternatively or additionally, lead 1500 can be configured to operably attach to one or more external devices, such as EPD 200 (e.g., when EPD 200 comprises a stimulator that is configured as a pacemaker, cardioverter, defibrillator, and/or any combination of two or more of these). Lead 1500 can be tunneled or otherwise routed from one or more locations in cardiac tissue, through the skin of the patient, to a location outside the body of the patient, for example, to attach to EPD 200 (e.g., when EPD 200 is at a location where EPD 200 is temporarily adhered to the skin of the patient). In some embodiments, one or more portions of EPD 200 are configured to be adhesively adhered to the skin, such as using adhesive 70. Alternatively or additionally, one or more portions of EPD 200 can be non-adhesively adhered to the skin (e.g., with a harness, strap, and/or other securing mechanism of system 10).

In some embodiments, system 10 includes one or more surgical or other tools, tool 90 shown, such as one or more tools for implanting a component of system 10 in a patient (e.g., in a surgical procedure, a percutaneous procedure, a laparoscopic or other minimally invasive procedure, and/or in any combination of two or more of these). Alternatively or additionally, tool 90 can comprise one or more devices for modifying a component of system 10, removing a component of system 10 from the patient, and/or adjusting one or more configuration parameters of a component of system 10. In some embodiments, tool 90 comprises a stylet, for example, a stylet used to implant lead 1500.

ID 100 can comprise controller 130, which can be configured to perform various functions of ID 100. Controller 130 can comprise a microprocessor, memory, and other components that can be constructed and arranged to control, perform, and/or otherwise enable one or more functions of ID 100. In some embodiments, controller 130 comprises one or more algorithms, algorithm 135 shown. In some embodiments, controller 130 comprises a memory for storing instructions to perform algorithm 135. Controller 130 can be constructed and arranged to execute algorithm 135 and to thereby execute one or more functions of ID 100. In some embodiments, each electrode 111 of electrode array 110 is independently addressable (e.g., electrically connected to at least two wires, such as ground and power or data, between each electrode and controller 130), such that signals (e.g., data and/or power) can be transmitted between controller 130 and each electrode 111 individually or collectively. Alternatively or additionally, controller 130 and/or electrode array 110 can be configured in a multiplexed arrangement, such that each electrode 111 can be individually addressed via a multiplexing component.

In some embodiments, controller 130 is configured to record electrical activity from one or more electrodes 111 (e.g., one or more electrodes 111 configured as sensing electrodes). Additionally or alternatively, controller 130 can be configured to provide stimulation signals to be delivered to the patient via one or more electrodes 111 (e.g., one or more electrodes 111 configured as pacing electrodes). In some embodiments, electrode array 110 comprises a set of electrodes 111 configured as pacing electrodes, and a set of electrodes 111 configured as sensing electrodes. Alternatively or additionally, controller 130 can be configured to alternate between pacing and sensing from an electrode 111 of electrode array 110 (e.g., in a multiplexed arrangement). In some embodiments, controller 130 is configured to simultaneously sense and pace from a given electrode 111. In some embodiments, multiple electrodes 111 can be multiplexed such as to sense (e.g., record signals) from one electrode 111 relative to a plurality of other electrodes 111 that collectively serve as a sensing reference. For example, the collective reference can be formed by the distance-weighted average of each of the electrodes 111 in the collected-reference (the collective “−” signal-reference) relative to the one measurement electrode (the one “+” signal-measurement). Additionally or alternatively, a similar arrangement can be provided for electrodes 111 delivering stimulation energy, such as when stimulation energy is delivered between a set of electrodes 111 (e.g., configured as an anode or a cathode) and a single electrode 111 (e.g., configured as a cathode or anode, respectively).

In some embodiments, ID 100 comprises a membrane or other material, coating 104, which can surround at least a portion of the surface of one or more components positioned on and/or within substrate 102. Coating 104 can comprise a biocompatible material, for example, a coating selected from the group consisting of: a silicone (PDMS) coating; a parylene coating; a water-based coating; a resin coating; a chemical coating; a steroidal coating; and combinations of these. Coating 104 can be configured to prevent irritation of the tissue onto which ID 100 is implanted, for example, to prevent an allergic reaction. In some embodiments, coating 104 comprises a bio-adhesive configured to permanently and/or semi-permanently adhere ID 100 to tissue (e.g., to the epicardial wall). For example, coating 104 can comprise a hydrogel (e.g., a hydrogel adhesive). Alternatively or additionally, system 10 can include adhesive 70, configured to be applied between ID 100 and tissue. In some embodiments, adhesive 70 is electrically conductive. In some embodiments, adhesive 70 comprises a UV activated adhesive. Adhesive 70 can comprise an injectable adhesive, for example, an injectable adhesive comprising a durometer under a threshold (e.g., a sufficiently soft adhesive). Adhesive 70 can comprise a biocompatible adhesive.

In some embodiments, ID 100 comprises one or more securing and/or stabilizing elements, anchoring element 105. Anchoring element 105 can be configured to secure, affix, stabilize, prevent (or at least limit) migration of, or otherwise prevent or limit unwanted motion of ID 100 (“secure” herein) before, during, and/or after implantation of device 100. Anchoring element 105 can be configured to temporarily anchor implantable device 100 (e.g., for a period of less than 1 month and/or less than 1 week) or to chronically anchor implantable device 100 (e.g., for a period of at least 1 month, at least 6 months, and/or at least 1 year). In some embodiments, anchoring element 105 comprises bioabsorbable materials. For example, anchor element 105 can comprise a bioabsorbable mesh that can be placed over one or more leads 1500 to temporarily hold lead 1500 in place. In some embodiments, anchor element 105 comprises a mesh (e.g., a bioabsorbable mesh) that functions as a replacement pericardial sac. In some embodiments, anchoring element 105 comprises a releasable and/or re-securable securing mechanism, such that ID 100 can be repositioned and/or removed (e.g., repositioned by a clinician using clinician device 300). Anchoring element 105 can be configured to interact with an anatomical feature to secure ID 100, such as by pushing against the pericardial sac to force ID 100 onto the epicardial wall. In some embodiments, anchoring element 105 comprises a material configured to promote tissue ingrowth and/or tissue overgrowth, such as to secure ID 100 as tissue growth interacts with anchoring element 105. For example, anchoring element 105 can comprise a fabric mesh.

In some embodiments, the various components of ID 100 are interconnected by one or more conduits, wires 112. In some embodiments, wires 112 comprise conductive routing filaments, for example, one or more conductive traces, such as one or more traces within and/or on a circuit board (e.g., a flexible circuit board). In some embodiments, wires 112 comprise traces within and/or on substrate 102. In some embodiments, electrode array 110 comprises one or more wires 112, for example, when electrodes 111 are electrically interconnected by wires 112. Wires 112 comprising conductive routings can each comprise a liquid metal routing, for example, a routing liquid phase eutectic gallium. In some embodiments, conductive traces are applied to substrate 102 (e.g., during a manufacturing process) with methods that include the manipulation of nanoparticles. For example, conductive traces can be formed such that wires 112 comprise nanowires consisting of graphene and/or silver. In some embodiments, wires 112 (configured as conductive traces of substrate 102) comprises a geometry configured to minimize Van der Waals, tensile, compressive, and/or other undesired forces, such as when wires 112 comprise a wavelike geometry (e.g., a sinusoidal geometry). The geometry of wires 112 can be configured such that wires 112 maintain a high level of conductivity, such when under strain.

ID 100 can include transceiver 120. Transceiver 120 can be configured to communicate (e.g., wirelessly communicate) with one or more other components of system 10, for example, one or more additional implanted devices 100′, as well as EPD 200, CD 300, console 400, and/or another component of system 10. Transceiver 120 can comprise a receiving and/or transmitting interface, antenna 125. Antenna 125 can be positioned on and/or embedded within substrate 102. In some embodiments, electrode array 110 comprises antenna 125, for example, when wires 112 of electrode array 110 are constructed and arranged to function as an antenna. Antenna 125 can comprise various shapes, for example, antenna 125 can comprise planar micro coils configured in various shapes.

ID 100 can include power module 140. Power module 140 can include one or more power-generating, power-harvesting, power-storing, power-transferring (e.g., via wireless power transfer) and/or other power-supplying components configured to deliver energy to ID 100. Power module 140 can be configured to provide power to one or more components of ID 100. In some embodiments, power module 140 comprises one or more batteries, capacitors, and/or other power-storing devices. In some embodiments, power module 140 comprises a solid-state battery, such as a miniature solid-state battery. In some embodiments, power module 140 comprises a rechargeable battery. In some embodiments, power module 140 comprises one or more capacitors. In some embodiments, power module 140 comprises at least one battery and at least one capacitor. In some embodiments, ID 100 does not include a battery (i.e., a source of power that is generated by an electrochemical reaction), a “battery-less design” herein, for example, when power module 140 is configured to harvest power (e.g., configured to harvest power transmitted wirelessly from EPD 200), and power module 140 is configured to store and directly provide the harvested power to power the various components of ID 100. Power module 140 can be constructed and arranged to “harvest” power from kinetic motion, for example, from kinetic motion of heart tissue when at least a portion of ID 100 is positioned on and/or within the heart. In some embodiments, power module 140 comprises one or more piezo electric components configured to convert kinetic energy to electrical energy.

In some embodiments, ID 100 can comprise patient sensor 160 shown. Patient sensor 160 can comprise one, two or more sensors selected from the group consisting of: an electrical sensor, such as a sensor configured to record an electrogram; a temperature sensor; accelerometer; position sensor; gravimetric sensor; pressure sensor; strain gauge; and combinations of these. System 10 can be configured to monitor one or more patient parameters based on information recorded by patient sensor 160, such as heartbeat, patient position, and/or patient activity.

ID 100 can include one or more functional elements, functional element 199 shown. Functional element 199 can comprise one, two, or more sensors selected from the group consisting of: pressure sensor such as blood pressure sensor; acoustic sensor; respiration sensor; gas sensor such as blood gas sensor; flow sensor such as blood flow sensor; temperature sensor; pH sensor; optical sensor; and combinations of these. In some embodiments, functional element 199 comprises one, two, or more transducers, such as an optical transducer (e.g., an LED).

System 10 can be configured to both monitor one or more patient parameters and to treat the patient based on the monitored parameters (e.g., based on an analysis of the monitored parameters). For example, system 10 can be configured to monitor (e.g., via electrode array 110) and analyze (e.g., via controller 130) electrograms recorded by ID 100, (e.g., unipolar and/or multipolar, for example, bipolar, modes of electrogram recording) and to pace and/or otherwise stimulate tissue if atrial fibrillation (AF) is detected. In some embodiments, system 10 is configured to monitor and/or record one, two, or more of electrophysiological activity, patient temperature, heartbeat information, and/or another patient parameter. In some embodiments, ID 100 is configured to stimulate tissue based on data recorded and/or analyzed by mapping module 420 of console 400. For example, mapping module 420 can be configured to identify irregular conduction patterns within one or more locations of cardiac tissue, as described herein, and to determine a set of stimulation parameters to be delivered by ID 100 to stimulate the tissue to treat (e.g., correct) the irregular conduction patterns.

In some embodiments, one or more portions of ID 100 (e.g., one, two, or more components of ID 100) can be bioabsorbable, biodegradable, and/or bioresorbable (“bioabsorbable” herein). For example, ID 100 can comprise a device including two or more electrodes 111 operably attached to antenna 125, that is configured to harvest RF energy (e.g., RF energy transmitted from EPD 200) and directly stimulate tissue by providing the harvested energy to electrodes 111 and electrodes 111, antenna 125 and/or the associated electrical traces of ID 100 can comprise a bioabsorbable conductive material, such as tungsten-coated magnesium (W/Mg). ID 100 can comprise one or more other components that comprise bioabsorbable magnesium. In some embodiments, electrodes 111, antenna 125, and/or the associated electrical traces of ID 100 are positioned on and/or within a bioabsorbable patch, such as a bioabsorbable patch configured to be attached to the epicardial surface with bioabsorbable suture.

External patient device 200 (EPD 200) can be constructed and arranged to be worn by the patient, such as when positioned on the skin of the patient (e.g., when EPD 200 is temporarily adhered or otherwise temporarily attached to the patient's skin), and/or when inserted in and/or otherwise attached to the patients clothing. Alternatively or additionally, EPD 200 can be held against the patient, such as when held against the patient's skin and/or clothing (e.g., by the patient and/or by a patient attachment device). For example, EPD 200 can be configured to be held against the patient, proximate ID 100, while EPD 200 communicates with ID 100 (e.g., for a brief period of time, such as less than 60 seconds). In some embodiments, EPD 200 includes attachment assembly 280. Attachment assembly 280 can include an adhesive, such as an adhesive patch, configured to adhere EPD 200 to the patient's skin for at least 6 hours, such as at least 12 hours, or at least 24 hours (e.g., before the adhesive patch must be replaced). Alternatively or additionally, attachment assembly 280 can comprise a harness, clip, specialized garment, or other non-adhesive based tool for positioning EPD 200 proximate the patient (e.g., proximate the location where ID 100 is implanted in the patient). For example, attachment assembly 280 can comprise a chest strap constructed and arranged to hold EPD 200 over the patient's heart, for example, when ID 100 is implanted onto the epicardial surface of the patient's left atrium. In some embodiments, EPD 200 comprises a device that is implanted subcutaneously or at another internal body location. Alternatively, one or more portions of EPD 200 are implanted in the patient and one or more portions are positioned external to the patient.

EPD 200 can include transceiver 220. Transceiver 220 can be configured to communicate (e.g., wirelessly communicate) with one or more components of system 10, for example, one or more implanted devices 100, and/or one or more additional external patient devices 200′, as well as CD 300, console 400, and/or other components of system 10. Transceiver 220 can comprise a receiving and/or transmitting interface, antenna 225. EPD 200 can be constructed and arranged to transmit power and/or data to one or more implantable devices 100, such as by transmitting a radio frequency (RF) energy from antenna 225, through the skin of the patient, towards ID 100, and ID 100 can be constructed and arranged to harvest the RF energy and/or receive the RF data via antenna 125 (e.g., a power-harvesting antenna). In some embodiments, EPD 200 is constructed and arranged to receive data from one or more implantable devices 100, such as when transceiver 120 is constructed and arranged to transmit RF data to EPD 200.

EPD 200 can include one or more user interfaces, user interface 250 shown. User interface 250 can include one or more user input and/or user output components, for example, one or more: displays, indicators (e.g., LEDs), speakers, buttons, microphones, and/or other user interface components. In some embodiments, EPD 200 includes one or more functional elements, functional element 299 shown. Functional element 299 can include one or more sensors and/or transducers. User interface 250 can display a visual representation of the heart chambers (e.g., a digital model) including one or more electrical conduction patterns (e.g., AF conduction patterns and/or sinus rhythm conduction patterns) that are displayed relative to the representation of the heart anatomy. In some embodiments, user interface 250 can display a representation of one or more portions of ID 100 (e.g., one or more electrodes 111) relative to the representation of the heart. In some embodiments, the conduction patterns displayed include pre-treatment and/or post-treatment (e.g., post pacing) conduction patterns. In some embodiments, the conduction patterns are displayed relative to each electrode 111 that is displayed on the representation of the heart. In some embodiments, user interface 250 can display various simulations of conduction patterns resulting from a proposed therapy to be delivered to treat the arrhythmia (e.g., AF) of the patient.

In some embodiments, functional element 299 of EPD 200 comprises one or more sensors that are used to record a patient parameter, such as a patient EEG. For example, functional elements 299 can comprise one, two, or more sensors (e.g., electrodes) that are positioned on EPD 200 such that the patient can place their thumbs or other fingers to contact the sensors, to provide an ECG recording (e.g., an additional ECG recording collected by system 10). For example, system 10 can perform diagnostic monitoring (e.g., ECG recording) on a predetermined schedule, but also allow for additional diagnostic monitoring (e.g., ECG recording) as determined by the patient (e.g., at any time). In some embodiments, the patient may choose to perform additional monitoring based on a physiologic condition, such as feeling dizzy, feeling faint, having palpitations, having shortness of breath, feeling tired, and the like. In some embodiments, the monitoring of the one or more patient parameters can be initiated by the patient. For example, the one or more patient parameters to be monitored (as initiated by the patient) can comprise at least an ECG, and the system can be configured to adjust the therapy provided (e.g., initiate stimulation energy delivery) based on detection of an arrhythmia via the monitored ECG. In some embodiments, the patient, clinician, and/or other user of system 10 can adjust the monitoring of one or more patient physiologic parameters, such as to establish a time-interval for monitoring of these parameters.

In some embodiments, functional element 299 of EPD 200, and/or another functional element of system 10, comprises one or more sensors that are configured to record EMG, EEG, and/or ECG, and system 10 is configured to analyze the recorded signals in order to perform a diagnosis and/or prognosis (“diagnosis” herein) of sleep apnea of the patient. EPD 200 can be configured to monitor one or more parameters related to the detection of sleep apnea selected from the group consisting of: movement, such as chest movement; snoring; body position; heart rate; O2 saturation; and combinations of these. In some embodiments, system 10 is configured to provide a sleep analysis. Analysis performed by system 10 (e.g., sleep apnea and/or other patient diagnosis such as a diagnosis of atrial fibrillation) can be accessible via an online portal (e.g., a patient portal hosted by server 600), and/or automated reports can be provided to the patient's managing physician.

EPD 200 can include processing unit 210 which can be configured to perform one or more functions of EPD 200. Processing unit 210 can include one or more algorithms, algorithm 215 shown. In some embodiments, processing unit 210 comprises a memory for storing instructions to perform algorithm 215. Processing unit 210 can be constructed and arranged to execute algorithm 215 and to thereby execute one or more functions of EPD 200. In some embodiments, processing unit 210 analyzes data (e.g., via algorithm 215) received from ID 100. For example, EPD 200 can receive data from ID 100, process (e.g., mathematically process) the information received via algorithm 215 (e.g., to determine if pacing should be performed, and to determine the parameters of stimulation energy to be delivered), and send information and/or power to ID 100 based on the processed information.

EPD 200 can include power module 240. Power module 240 can include one or more power-generating, power-harvesting, power-storing, and/or other power-supplying components configured to deliver energy to EPD 200, and/or to deliver power to ID 100 via wireless power transfer. Power module 140 can be configured to provide power to one or more components of EPD 200. In some embodiments, power module 240 comprises one or more batteries, capacitors, and/or other power-storing devices. Power module 240 can be constructed and arranged to “harvest” power from kinetic motion. In some embodiments, power module 140 comprises one or more piezo electric components configured to convert kinetic energy to electrical energy.

CD 300 can include one or more catheters and/or or one or more surgical tools for delivering ID 100 into the patient. Additionally, CD 300 can include one or more devices configured to diagnose and/or treat the patient, such as to perform a diagnosis and/or a treatment during a clinical procedure in which ID 100 is implanted into the patient. For example, CD 300 can comprise a cardiac mapping catheter which can be used to collect data (e.g., data to be processed by console 400) such as to map the cardiac electrical activity of the heart. Additionally or alternatively, CD 300 can comprise an ablation catheter which can be used to ablate tissue (e.g., cardiac tissue). In some embodiments, system 10 can include one or more clinician devices 300 that are constructed and arranged to enable the clinician to perform: a mapping procedure, a tissue treatment procedure (e.g., an ablation procedure or other tissue treatment procedure), and/or an ID 100 implantation procedure (e.g., for continued, post procedural treatment of the patient).

In some embodiments, CD 300 comprises electrode array 310 shown, which can comprise one or more arrays of electrodes that can be inserted into the patient. Electrode array 310 can comprise one or more electrodes 311. CD 300 can include user interface 350 shown. User interface 350 can include one or more user input and/or user output components, for example, one or more: displays, indicators (e.g., LEDs), speakers, buttons, levers, microphones, and/or other user interface devices. In some embodiments, user interface 350 comprises a handle (e.g., a catheter handle) including one or more controls, such as a steering control.

In some embodiments, CD 300 includes transceiver 320. Transceiver 320 can comprise an assembly configured to communicate (e.g., wirelessly communicate) with one or more components of system 10, for example, one or more implanted devices 100, one or more external patient devices 200, console 400, and/or other components of system 10. Transceiver 320 can comprise a receiving and/or transmitting interface, antenna 325. In some embodiments, CD 300 includes one or more functional elements, functional element 399 shown. Functional element 399 can include one or more sensors and/or transducers.

In some embodiments, system 10 includes a data storage and processing device, server 600. Server 600 can comprise an “off-site” server (e.g., outside of the operating room or other clinical site in which ID 100 is implanted), such as a server maintained by the manufacturer of system 10. Alternatively or additionally, server 600 can comprise a cloud-based server. Server 600 can include processing unit 610 shown, which can be configured to perform one or more functions of server 600. Processing unit 610 can include one or more algorithms, algorithm 615. In some embodiments, processing unit 610 includes a memory for storing instructions to perform algorithm 615. Processing unit 610 can be constructed and arranged to execute algorithm 615 and to thereby execute one or more functions of server 600. Server 600 can be configured to receive and store various forms of data, such as: patient, procedural, device, and/or other information, data 620. Data 620 can comprise data collected from multiple patients (e.g., multiple patients treated with system 10), such as data collected during and/or after clinical procedures where ID 100 was implanted into the patient. For example, data can be collected from ID 100, transmitted to EPD 200, and sent to server 600 for analysis. In some embodiments, one or more devices of system 10, such as EPD 200 and server 600, can communicate over a network, network 50, for example, a wide area network such as the Internet. In some embodiments, system 10 includes a virtual private network (VPN) through which various devices of system 10 transfer data.

Algorithm 615 can be configured to analyze data 620. For example, algorithm 615 can be configured to analyze data 620 collected from multiple patients to identify similarities and/or differences in treatment parameters and patient results. In some embodiments, algorithm 615 comprises a machine learning and/or other artificial intelligence algorithm (“AI algorithm” herein) that can be configured to identify patterns in the correlations between treatment parameters and results based on data collected from multiple patients. In some embodiments, algorithm 615 analyzes patterns to determine better treatment parameters for one or more patients to be treated using system 10. For example, algorithm 615 can identify one or more patterns in the data (e.g., one or more patterns associated with efficacy of the treatment being delivered to the patient) by analyzing data 620 collected from many patients (e.g., tens of thousands of patients). Algorithm 615 can be further configured to use these patterns to determine whether a patient (e.g., in the set of patients from which the data was collected and/or in a new patient) is receiving sub-optimal treatment (e.g., the parameters associated with pacing and/or other energy being delivered could be modified to improve efficacy). System 10 (e.g., via algorithm 615) can be configured to alert the clinician of a patient receiving sub-optimal treatment, and to recommend (e.g., via CD 300, such as the clinician's phone or computer) the parameters to be adjusted. In some embodiments, the clinician may schedule an appointment to adjust the parameters (e.g., in person), or the parameters can be adjusted remotely, for example, when CD 300 is configured to adjust the parameters remotely via network 50. Alternatively or additionally, server 600 can adjust the parameter automatically (e.g., via network 50). In some embodiments, one or more parameters are automatically adjustable (e.g., within certain thresholds), while other parameters require clinician approval.

As described herein, system 10 can comprise one or more algorithms, such as algorithms 135, 215, 415 and/or 615 shown in FIG. 1. Various algorithms of system 10 (e.g., algorithms 135, 215, 415, and/or 615) can be referred to singly or collectively herein as algorithm 500. In some embodiments, algorithm 500 comprises a machine learning and/or other artificial intelligence algorithm (“AI algorithm” herein). Any algorithmic process described herein may be performed by any algorithm of system 10 (e.g., algorithms 135, 215, 415, and/or 615). The various processors and/or controllers of system 10 can each comprise memory configured to store instructions for performing the algorithms described herein.

In some embodiments, algorithm 500 can comprise a set of algorithms configured to identify the presence of atrial fibrillation and deliver (e.g., automatically deliver via ID 100) pacing stimuli across a spatially distributed array of electrodes placed on the left atrium (e.g., electrodes 111 of electrode array 110). The pacing stimuli delivered by ID 100 can be imperceptible to the patient. The pacing stimuli can be precisely timed at each electrode 111 to advance and/or block fibrillation wavefronts. Delivery of pacing stimuli by ID 100 can be configured to synchronize atrial activation to the pattern of stimulation and can be configured to automatically stop upon restoration of normal rhythm.

System 10 can be configured to record electrical activity, such as cardiac electrical activity, and algorithm 500 can be configured to analyze the recorded electrical activity. For example, electrical activity can be recorded via one or more electrodes 111 of electrode array 110. The recorded electrical activity can be transmitted, via transceiver 120, to EPD 200. Algorithm 215 of EPD 200 can be configured to analyze the received data, and to determine if stimulation is required to treat the patient. Algorithm 215 can determine a set of stimulation parameters to be delivered by ID 100 based on the received electrical data (e.g., based on a recorded pattern of conduction within the cardiac tissue). For example, algorithm 215 can determine the location and instances in time to deliver stimulation energy (e.g., via electrodes 111). Alternatively or additionally, the recorded electrical data can be transmitted to console 400 and/or server 600, such that algorithms 415 and/or 615 can analyze the data and determine stimulation parameters. The stimulation parameters determined by algorithm 500 can be transmitted back to implantable device 100, via transceiver 220, to ID 100. In some embodiments, the stimulation parameters prescribe stimulation pulses to be delivered as a sequence of pulses to be delivered simultaneously and/or asynchronously, and/or regularly and/or irregularly. The stimulation pulses can be delivered from one or more electrodes 111. In some embodiments, algorithm 135 is configured to process stimulation parameters received from EPD 200 and stimulate via electrodes 111 based on the processed parameters. Alternatively or additionally, ID 100 does not comprise an algorithm, and is configured to stimulate based on power and/or data received from EPD 200 (e.g., stimulation power is received by transceiver 120 and provided to an electrode 111 based on data received with the transmitted power).

In some embodiments, system 10 is configured to stimulate cardiac tissue by providing electrical stimulation such that any pain and/or discomfort caused by the delivery of the electrical stimulation is below a perception threshold (e.g., the patient doesn't feel any pain or discomfort caused by the delivery of the electrical stimulation).

As described herein, system 10 can be configured to perform multisite pacing, such as to terminate AF and/or SVT of the patient. AF can be caused by a stretch-induced infiltration of fibrosis that is progressively and broadly distributed across the left atrium. Global, simultaneous mapping of AF has revealed patient-specific confined zones of conduction that are distributed primarily across three anatomical regions of the left atrium: (1) posterior wall; (2) anterior-roof; and (3) anterior-septum. In the early phase of AF, categorized as “paroxysmal”, the progression of fibrosis is more confined to the muscular sleeves surrounding the pulmonary veins and the posterior wall of the left atrium. As the disease of AF progresses into the “persistent” stage, fibrosis spreads beyond the posterior wall, predominantly emerging at patient-specific locations across the roof and septum, anteriorly.

The feasibility of low-voltage shocks and multisite pacing for terminating AF can be limited by: (1) the number, size, and/or distribution of electrodes placed about the left atrium; and (2) the pattern of stimulation energy delivered. The progressive nature of the disease requires matching the spatiotemporal characteristics of pacing with the patient-specific distribution of fibrosis.

In the early, paroxysmal phase of AF, pacing can be delivered from multiple (e.g., 3 or 4) electrodes distributed within the Vein of Marshall and the adjacent coronary sinus. These locations are close to the lateral border of the posterior wall and the left pulmonary veins, where stimulation is required for effective interruption of fibrillatory conduction in the region of the left atrium that is relevant for paroxysmal AF.

In the later persistent and long-standing phases of AF, pacing can be delivered from more electrodes (e.g., 5 or 6 electrodes) that are distributed epicardially on the posterior wall, the anterior roof, and/or superior septum. These locations are close to the critical, “confined zones” of conduction that maintain AF. Stimulation is required to be delivered near these zones for effective interruption of fibrillatory conduction in the regions of the left atrium that are relevant for persistent AF.

In some embodiments, one or more sensors (e.g., functional element 199 comprising one or more sensors and/or one or more electrodes 111 configured as a sensor) of ID 100 (e.g., an ID 100 comprising one or more implantable devices) are positioned at one or more locations proximate heart tissue and are configured to produce signals from which a calculation of pressure within a chamber (e.g., pressure of the blood within the left atrium) can be determined (e.g., by one or more of algorithms 500), such as is described in reference to FIG. 1 herein. In some embodiments, the signals are recorded from (e.g., ID 100 and the associated sensors are implanted at) a location outside of the chamber of the heart for which the chamber pressure is determined (e.g., outside of the left atrium when left atrial pressure is determined). For example, one, two or more electrodes 111 of ID 100 (e.g., an ID 100 comprising one, two, or more implantable devices) can be configured to record signals to determine a chamber pressure, such as when an impedance measurement is performed to identify impedance characteristics of tissue surrounding a heart chamber (e.g., the left atrium) that can be correlated (e.g., by an algorithm 500) to the chamber pressure (e.g., left atrial pressure). Alternatively or additionally, functional element 199 of ID 100 (e.g., an ID 100 comprising one, two, or more implantable devices) can comprise one, two, or more sensors configured to produce a signal (e.g., record a physiologic parameter) that can be used to determine the pressure of a heart chamber. In some embodiments, at least one, two, or more of these sensors are positioned within the chamber for which the chamber pressure is determined. In some embodiments, at least one, two, or more of these sensors are positioned on tissue that is proximate, but outside the chamber for which the chamber pressure is determined (e.g., on the epicardial wall), such as when no sensors are present within that chamber. Applicable sensors for producing a signal used by system 10 (e.g., an algorithm 500) to determine a chamber pressure include but are not limited to: a pressure sensor; a strain gauge (e.g., to measure strain in tissue that can be correlated to the chamber pressure proximate the tissue on which the sensor is positioned); an accelerometer (e.g., to measure tissue motion that can be correlated to chamber pressure proximate the tissue on which the sensor is positioned); an ultrasound sensor and/or other acoustic sensor, such as a doppler ultrasound sensor (e.g., configured to measure one or more blood flow parameters which can be correlated to the chamber pressure); an optical sensor (e.g., configured to measure one or more tissue and/or blood properties which can be correlated to the chamber pressure); and combinations of these.

In some embodiments, system 10 is configured to deliver pacing stimulation during sinus rhythm, such that the stimulation is configured to synchronize activation of the left ventricle. This stimulation can improve the timing and volume filling of the left ventricle and can increase cardiac output. Since the 1990's, it has been shown that a natural variability in heart-rate reduces vulnerability of the cardiac substrate to initiation and/or re-initiation of arrythmia. Nonlinear pacing can be defined as delivery of pacing energy that is irregular, aperiodic, and/or otherwise varying (e.g., in level, frequency, modulation, and the like). System 10 can be configured to deliver nonlinear pacing. Alternatively or additionally, system 10 can be configured to deliver spatiotemporal resynchronization therapy, SRT. SRT shall include the ability to control a fibrillating substrate by deterministically pacing into the narrowed excitable gap present during AF, such as from a well distributed set of electrodes. Once each electrode has gained control of the adjacent substrate, system 10 selectively advances the pacing to achieve alignment across electrodes, prolonged, and inhibited to allow normal sinus rhythm to return. Accordingly, after delivery of SRT via system 10 has terminated AF, another algorithm (e.g., algorithm 135) can be applied to device 100 in which the baseline sinus rhythm is nonlinearly (e.g., deterministically) varied with “irregularly-early” pacing pulses that impose said deterministic variation in heart rate. In another embodiment, this can be achieved by first deriving the mean and standard deviation (or median and IQR) of heart rate for a predetermined period. Based on these parameters, stimuli can then be delivered according to a “fractal” or other appropriate nonlinear function that paces the heart at a time that is “earlier” than the mean cycle-length (e.g., inverse of heart-rate) to deterministically impose a variation in said beat, as compared to the previous beat. Similarly, subsequent beats can be at differing durations of “earliness” to impose a desired variability in the heart rate over time. Such a configuration can also include occasional inhibition of pacing to achieve the “intrinsically-longest” cycle-length, as a part of the overall range of variation that occurs over time. Such a configuration can also include periodic cessation of pacing to re-assess the mean and standard deviation (or median and IQR) of heart rate. In such a configuration, the algorithm (e.g., algorithm 135) can follow the natural variation in heart rate and enhance the natural variation (or lack thereof) with “variably-early stimulation”. The overall goal of such a pacing algorithm (e.g., algorithm 135) is to achieve a level of variation that optimizes the probability of reducing vulnerability to initiation and/or re-initiation of arrhythmia. In some embodiments, system 10 can deliver multiple different forms of energy delivery, such as to treat different medical conditions of the patient (e.g., at least AF).

During AF ablation procedures, AF is terminated into sinus rhythm during the delivery of ablation in approximately 35% of procedures. In approximately 10% of these 35% of procedures, the SA-node fails to automatically re-initiate a baseline (“normal”) sinus rhythm. In such cases, the SA-node appears to have been electrically remodeled into a quiescent state that is presumably due to the rapid impingement of activation upon it during the ongoing AF. It has also been observed that such instances of cessation are temporary, with SA-node activation gradually “waking-up” and resuming the maintenance of baseline sinus rhythm. Such wake-up periods generally range from a few minutes to about 30 minutes. At this point in the procedure, the laboratory stimulator is applied to address the bradycardia and maintain a normal baseline heart rate, while the SA-node is recovering its ability to maintain sinus rhythm. This is performed by the support staff (e.g., laboratory) at the request of the clinician (e.g., physician), by pacing through existing catheter-electrodes that are already placed in the heart. Accordingly, after nonlinear pacing (e.g., spatiotemporal resynchronization therapy, SRT, as defined herein) via system 10 has terminated AF, another algorithm (e.g., algorithm 135) can be applied by ID 100 in which the baseline sinus rhythm is temporarily maintained at a “typically normal” rate. In another embodiment, this can be achieved by first deriving the mean heart rate over a short period (e.g., several beats). In accordance with a maximum threshold of cycle-length, stimulation can be delivered (including “immediately”) in the “AAI” pacing mode (Atrial sensing/Atrial pacing/Inhibited). Such a configuration can comprise periodically inhibiting pacing, while pacing is inhibited re-assessing the intrinsic heart rate and determining if the SA-node has recovered; if recovery is determined, pacing can remain inhibited. Additionally, such a configuration can also include variation in the pacing rate, as disclosed hereinabove, with the goal of reducing the vulnerability of the cardiac substrate to initiation and/or re-initiation arrhythmia. Another embodiment can consider application of the VVI pacing mode (Ventricular sensing/Ventricular pacing/Inhibited). This mode can be considered less desirable than atrial pacing for aiding in the recovery of the SA-node, as it depends on adequate retrograde conduction through the AV-node. Conversely, atrial pacing is directly “in-line” with the SA-node, and such conduction characteristics may play a positive role in recovery of the SA-node. Ventricular pacing, on the other hand, directly addresses the undesired slow heart rate (e.g., bradycardia) without any consideration on the “health” of AV-node conduction. Regardless, ventricular pacing is inherently less desirable than atrial pacing, as an additional device (e.g., ID 100) must be applied upon the ventricle to fulfill this embodiment. In the rare instance where the SA-node remains quiescent for a prolonged period, then the clinician (e.g., physician) can be notified through the device upload. In such rare cases, the patient enters a separate category that requires another type of therapy. In that case, there would likely be implantation of a pacemaker to address bradycardia caused by a “sick sinus node”. In the less-rare instance of AV-node conduction disease, the patient may have been identified as having various levels of AV-block at a much earlier time in their treatment history. In that case, implantation of a pacemaker may likely have already been performed to address bradycardia caused by abnormal AV-node conduction. In either case, as described hereinabove, implantation of a pacemaker can be symbiotic with implantation with ID 100.

In some embodiments, system 10 is configured to provide treatment for left atrial pre-conditioning (e.g., pre-conditioning for a patient with atrial fibrillation prior to an ablation procedure). Pre-ablation pacing therapy, implanting the device 3 months prior to ablation to maximize the likelihood of maintaining sinus rhythm after the ablation procedure. Delivered over some months, pre-ablation pacing can result in enough reverse-remodeling to regain some level of organization that reveals demarcated, putative ablation targets in a future ablation procedure. In some embodiments, system 10 is configured to provide treatment for left atrial re-conditioning, for example, by providing a “blanking period” post ablation. Early recurrence of atrial fibrillation (AF) or atrial tachycardia (AT) after catheter ablation (CA) in AF patients is known to be a transient phenomenon. An expert consensus group recommends to measure the success of an ablation therapy from 3 months after the procedure onward. This period is termed the “blinding or blanking period” and is now widely adopted in both clinical routine and clinical trials. The 3-month period is often thought of as a time for the lesions to maturate and heel.

The underlying pathophysiological processes responsible for early recurrence and the delayed cure are unknown. Early recurrence is considered the consequence of ablation-induced proarrhythmic factors that are limited to the time frame of the blanking period. Whereas a delayed cure may also be the cause of an antiarrhythmic effect that develops in the course of the blanking period and that is not necessarily related to electrical isolation of the PV. A potential confounding influence is played by the use of AADs during the blinding period, as they may paradoxically act as a possible proarrhythmic factor. AADs are usually discontinued after the 3-month period, and this may unmask the antiarrhythmic effect of the ablation procedure. The theoretical basis of the blanking period is based on such observations. However, the clinical implications of early recurrence may be avoided. The incidence of early AF recurrence during the 3-month blinding period following PVI ranges from 9 to 65%. It has been shown that 54% of patients have early recurrence within week 1 to 2 following ablation therapy, after which the percentage drops to 38% in weeks 2 to 4 and to 24% in weeks 4 to 6. The first episode of early recurrence occurs within month 1 of the blinding period in 81% to 91% of patients with early recurrence. This emphasizes the dynamic nature of the blinding period.

AF is a complex arrhythmia with multiple possible mechanisms underlying initiation and maintenance. Ablation is successful in 60% of paroxysmal AF patients. AF can recur during the 3-month blanking period after ablation. No therapy or interaction takes place during this 3-month blanking period.

Recent retrospective studies indicate that right atrial pacing decreases the incidence of AF They demonstrated significant benefit for atrial pacing with improved survival and a decreased incidence of thromboembolic events, AF, and congestive heart failure, especially after longer follow-up. The hypothesis: if during the so-called blanking period, where no therapy or patient management is occurring, change this 3-month period into a left Atrial “Re-Conditioning” period. System 10 can be configured to perform continuous (7/24) sinus rhythm pacing. This not only keeps the post ablation patient in sinus rhythm but reverse remodels the left atrium (AF remodels the left atrium to AF), reconditioning reverses this AF remodeling back to normal. Post the 3-month LA re-conditioning, the ID 100 can perform normal monitoring and deliver therapy only when the patient has an AF episode.

In some embodiments, system 10 is configured to deliver multisite pacing for termination of atrial fibrillation, such as is described herein. AF is caused by a stretch-induced infiltration of fibrosis that is progressively and broadly distributed across the left atrium. Global, simultaneous mapping of AF has revealed patient-specific confined zones of conduction that are distributed primarily across three anatomical regions of the left atrium: (1) posterior wall; (2) anterior-roof; and (2) anterior-septum. In the early phase of AF, categorized as “paroxysmal”, the progresssion of fibrosis is more confined to the muscular sleeves surrounding the pulmonary veins and the posterior wall of the left atrium. As the disease of AF progresses into the “persistent” stage, fibrosis spreads from the posterior wall predominantly across the roof and septum, anteriorly, toward the mitral valve annulus. The feasibility of low-voltage shocks and multisite pacing for terminating AF has been demonstrated and is primarily limited by: (1) the number and distribution of electrodes placed about the left atrium; and (2) the algorithm (e.g., algorithm 135) governing the pattern of stimulation. The progressive nature of the disease requires matching the spatiotemporal characteristics of pacing with the patient-specific distribution of fibrosis. In the early, paroxysmal phase of AF, pacing is delivered from 3 or 4 electrodes distributed within the Vein of Marshall and the adjacent coronary sinus. These locations are close to the lateral border of the posterior wall and the left pulmonary veins, whereby stimulation is required for effective interruption of fibrillatory conduction in the region of the left atrium that is relevant for paroxysmal AF. In the later, persistent and long-standing phases of AF, pacing is delivered from 5 or 6 electrodes distributed epicardially on the posterior wall and/or the anterior roof. These locations are close to the critically, “confined zones” of conduction that maintain AF. Stimulation is required to be delivered near these zones for effective interruption of fibrillatory conduction in the regions of the left atrium that are relevant for persistent AF.

In some embodiments, system 10 is configured to provide left atrial pacing therapy to improve hemodynamic function. Extensive ablation of the left atrium for treatment of AF can lead to a decrease in overall hemodynamic function. This occurs when significant delay of conduction to the left-atrial appendage is imposed by ablation lesions that are delivered between the insertion of Bachmann's Bundle, in the high septum and roof, and the appendage. Normally, the appendage is a significant contributor to the mass transport of blood from the left atrium to the left ventricle. If activation of the appendage is delayed by intervening ablation-lesions, then the timing of active pumping by the appendage is also delayed. It is possible for this delay to be long enough to be working against the closure of the mitral valve during the beginning of left ventricular contraction. In this case, filling of the left ventricle is incomplete and overall hemodynamic performance is compromised. In the long term, this effect, in combination with other factors, can lead to the gradual decline of heart failure.

System 10 can be configured to enable the placement of one or more pacing electrodes in the distal-reach of the Vein of Marshall. This region of the vein is in relatively close apposition to the posteromedial aspect of the left atrial appendage. Specifically, this is the same general location where the left-lateral branch of Bachmann's Bundle terminates, and which enables timely activation of the left atrial appendage. Accordingly, after pace termination of AF by system 10, the most distal electrode in the Vein of Marshall can then be used to ensure timely activation of the left atrial appendage. This concept is analogous to ventricular resynchronization therapy, whereby the right and left ventricles are paced in specific locations and with relative timing of stimulation that are intended to synchronize activation of both ventricles for the purpose of improving and optimizing overall hemodynamic performance. In the case of delayed conduction in the left atrial appendage, the effectiveness of ventricular resynchronization therapy can be compromised or severely limited. Accordingly, left atrial appendage resynchronization pacing can improve overall hemodynamic performance, either with or without ventricular resynchronization therapy. One embodiment can include sensing the timing of activation from one or more implanted electrodes 111 and/or one or more surface ECG leads. These signals would be used to algorithmically (e.g., via algorithm 135) sense the relative timing between the beginning of left atrial activation and the time of activation of the left atrial appendage. The algorithm (e.g., algorithm 135) can command an electrode 111 to pace at an optimal time in the attempt to promote optimal left-ventricular filling.

In some embodiments, system 10 is configured to deliver median and/or ulnar nerve stimulation for the purpose of shifting the operating-point of afferent autonomic tone toward inhibition of AF across the left-atrial substrate. Additionally or alternatively, system 10 can be configured to deliver stimulation to nerve bundles in the feet or in the ear. The autonomic nervous system plays a significant role in modulating the overall state of syncytial-conduction throughout the left-atrial myocardial substrate. It is challenging to access both the efferent nerve fibers, from the gangliated plexi, as well as the distribution of afferent fibers that insert throughout the left-atrial chamber. Consequently, the objective of either ablating (e.g., destroying) these nerves or modulating (e.g., stimulating) these nerves has not been adequately effective. It has been shown acupuncture therapy that targets the Median Nerve promotes cessation of arrhythmias, notably AF. Recent clinical studies have validated this observation from the practice of acupuncture. System 10 can be extended and/or coordinated, algorithmically (via algorithm 135), together with sensing of intracardiac and/or surface ECG signals to stimulate pacing electrodes that are located on the arm, for example, within a wrist-band, that is positioned to stimulate the Median and/or Ulnar nerves and thereby mediate modulation of autonomic vagal tone in the direction of at least partial inhibition of AF.

In some embodiments, system 10 includes one or more computer applications (e.g., software applications performed by a processor of system 10, where the instructions for performing the applications are stored in memory of system 10), such as cardiac simulator 4101, comprising a cardiac tissue simulator application. Cardiac simulator 4101 can include an interactive, physiologically realistic, and accurate computer simulation that allows multiple scenarios to be tested in a “live” environment (e.g., during a clinical procedure). In some embodiments, cardiac simulator 4101 is performed by processing unit 410 of console 400. Cardiac simulator 4101 can be used for the rapid development and testing of treatment strategies for atrial fibrillation (AF). Cardiac simulator 4101 can include a model of cardiac tissue that allows the user to define the regions and zones of anisotropic conduction to test the effectiveness of a variety of AF treatment strategies.

The model was developed using standard electrophysiologic (EP) parameters established in the literature. An advanced implementation of the Fitzhugh-Nagumo model reproduces the action potential morphology of the human atrium enabling live simulations. To reproduce the complex conduction patterns (CCP) of AF identified in the EP lab, a bi-layer model was implemented to represent epi- and endocardial dissociation. The model allows the user to define regions of fibrosis, zones of slow conduction, and action potential duration (APD). The user can graphically draw these regions and zones, including gradients assigned by maximum and minimum values of conduction velocity (CV) and APD. The EP lab experience and workflow is reproduced, with CCP visualized on a 3D anatomy and signal traces of calculated potentials displayed. To test the CCPs and performance of the model, isolated, geometrically, symmetric zones of conduction were defined on a 3D left atrial model.

Referring additionally to FIG. 1A, a partial anatomic, partial schematic view of a system of devices is illustrated, consistent with the present inventive concepts. In some embodiments, system 10 includes one, two, three, or more implantable devices, such as the two implantable devices 100a and 100b shown. For example, system 10 can include ID 100a, including an implantable device positioned proximate the left atrium of the heart, for example, on the epicardial surface of the left atrium or within a cardiac vessel proximate the left atrium. ID 100a can comprise one or more electrodes, electrode 111a shown, which can be similarly positioned on the epicardial surface of the left atrium and/or within a cardiac vessel proximate the left atrium. System 10 can also include ID 100b, including an implantable device with at least a portion positioned away from the heart, for example, comprising an implantable pulse generator (IPG) that is implanted in a subcutaneous pocket. In some embodiments, ID 100b includes a lead operably connecting controller 130b to one or more electrodes, electrode 111b shown, and/or antenna 125b implanted proximate the heart, for example, implanted within the right atrium on the septal wall. Antenna 125b can be positioned proximate antenna 125a of ID 100a to minimize the transmission distance between ID 100a and ID 100b (e.g., the distance over which power is transmitted from ID 100b to ID 100a).

In addition to one, two, three, or more internal devices, system 10 can include one, two, three, or more external patient devices, such as the four external devices 200a-d shown. For example, system 10 can include EPD 200a, including a patient worn device configured to be positioned (e.g., temporarily positioned by the patient) proximate an implantable device, such as proximate ID 100b as shown, and/or proximate ID 100a (e.g., when system 10 does not include a subcutaneous implant, such as ID 100b). EPD 200a can be positioned to minimize the transmission distance between transceiver 220a and the intended ID (e.g., ID 100a and/or ID 100b). System 10 can also include one or more additional patient worn and/or handheld devices, such as EPD 200b comprising a wrist worn device (e.g., a smart watch), EPD 200c comprising an ankle worn device (e.g., a “smart” sock, and/or a fitness tracker), and/or EPD 200d comprising a computing device (e.g., a smartphone). In some embodiments, EPD 200a,b,c,d are configured to communicate with each other, for example, when one EPD 200 (e.g., EPD 200d) is configured to gather data collected by each EPD 200 and/or ID 100 and to analyze the aggregated data (e.g., via algorithm 215). In some embodiments, and EPD 200 is configured to collect data and send the aggregated data to server 600 (e.g., when EPD 200d comprising a smart phone is configured to collect all patient data recorded by system 10 and transmit the data via the internet to server 600 for analysis).

In some embodiments, one or more of the devices of system 10 comprises two or more batteries, such as when a device includes a primary power supply (e.g., one or more batteries and/or one or more capacitors) and a backup power supply (e.g., one or more batteries and/or one or more capacitors). For example, power module 140 of ID 100 can comprise a first battery and/or capacitor, battery 1401, and a second battery and/or capacitor, battery 1402 (as shown in FIG. 1). Battery 1401 can be configured as a main power source for ID 100, and battery 1402 can be configured as a backup power source, for example, to be used if battery 1401 is depleted. In some embodiments, battery 1401 and/or 1402 are rechargeable. In some embodiments, battery 1402 is configured to maintain operation of ID 100 if the patient is unable to charge battery 1401 for a period of time (e.g., the patient is away from the charger when battery 1401 runs low on power). Batteries 1401 and 1402 can comprise batteries with similar or dissimilar battery chemistry, for example, lithium-ion batteries and/or lithium thionyl chloride batteries. In some embodiments, ID 100 comprises a single battery 1401, and system 10 (e.g., algorithm 135 of system 10) is configured to virtually partition the single battery into two virtual batteries, such that ID 100 functions as if battery 1401 comprised a main battery and a backup battery. In some embodiments, batteries 1401, 1402 comprise different size batteries (e.g., batteries with different amp-hour capacity). In some embodiments, ID 100 can operate in two or more power modes, for example, a first, “normal-power” mode, and a second, “low-power” mode. In some embodiments, when the main power supply of ID 100, battery 1401, is depleted, ID 100 transitions into the low power mode and operates using energy from battery 1402, for example, until battery 1401 can be recharged. In some embodiments, if battery 1402 is used for a period of time, for example, at least 1 hour, at least 6 hours, at least 12 hours, at least 1 day, and/or at least 1 week, ID 100 and/or battery 1402 (e.g., when battery 1402 is replaceable), are replaced (e.g., in a follow-up procedure) to ensure ID 100 maintains adequate backup power supply (e.g., backup battery supply and/or backup capacitor supply) at all times. In some embodiments, power module 140 is configured to periodically test battery 1401 and/or 1402, for example, to confirm battery 1402 is charged and available to provide backup power. In some embodiments, system 10 can be configured to enter an alert mode (e.g., provide an alert to the user as described herein) if a power supply level (e.g., battery level and/or capacitor charge level) is below a threshold, and/or if ID 100 transitions to a low power mode.

In some embodiments, ID 100 is configured to deliver both life-saving therapy (or life-sustaining therapy, either or both “life-saving therapy” herein) as well as quality-of-life therapy. In these embodiments, ID 100 can be configured to provide multiple forms of therapy, such as a first form of therapy (e.g., a life-saving therapy) and/or a second form of therapy (e.g., a quality-of-life therapy), and ID 100 can be configured to deliver (e.g., only deliver) a particular type of therapy based on the amount of energy stored in ID 100 (e.g., the energy stored in battery 1401 and/or battery 1402). In these embodiments, ID 100 can be configured to provide only life-saving therapy when the energy stored in battery 1401 and/or battery 1402 is below a threshold (e.g., below a pre-determined amount of energy). In some embodiments, ID 100 can be configured to provide life-saving therapy comprising cardiac pacing (e.g., pacing of the ventricle, such as when complete AV node block is present), and ID 100 can be further configured to provide quality-of-life therapy comprising providing stimulation energy to treat AF. In some embodiments, ID 100 is configured to deliver atrial pacing that is configured to: address bradycardia due to sinus node dysfunction; maintain normal heart rate variability; and/or to reduce the vulnerability to re-initiation of AF. ID 100 can also be configured to perform non-therapy-related tasks, as described herein, such as communicate with external devices (e.g., EPD 200), perform self-diagnostics, monitor patient parameters, and/or other non-life-saving tasks. In some embodiments, where ID 100 is configured to operate (e.g., sequentially operate) in a regular-power mode and a low-power mode, when ID 100 enters a low-power mode, only life-saving therapy is provided to the patient, such as to allow ID 100 to continue to operate (e.g., as long as possible) until a normal or otherwise improved power mode can be re-initiated (e.g., when battery 1401 is charged). Alternatively or additionally, ID 100 can comprise a first battery and/or capacitor, battery 1401, that is used to power a first set of operations, and a second battery and/or capacitor, battery 1402, that is used to power a second set of operations. For example, battery 1401 can be used to power life-saving operations, such as cardiac pacing (e.g., when ID 100 is configured as a pacemaker), and battery 1402 can be used to power quality-of-life therapy operations, for example, operations of ID 100 which monitor for and treat AF.

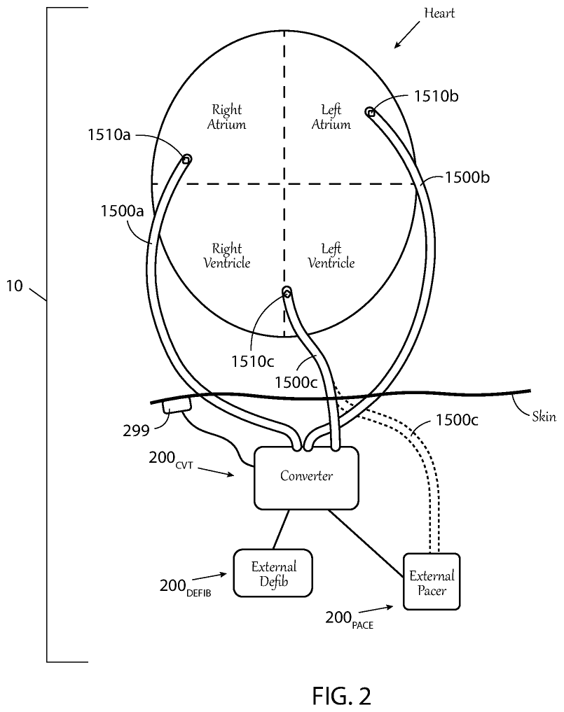

Referring now to FIG. 2, a schematic illustration of a system for providing post operative treatment of AF is illustrated, consistent with the present inventive concepts. System 10 of FIG. 2 can be of similar construction and arrangement to system 10 described in reference to FIG. 1 and otherwise herein. Although predictors exist for identifying AF, such an identification process is challenging. Accordingly, in some embodiments, system 10 is intended to include implants to be placed into any post operative cardiac patient (e.g., not just those patients that have a history of AF). In some embodiments, one or more leads of system 10, leads 1500, are implanted (e.g., placed with or without the intent of subsequent removal) proximate the patient's heart. The leads 1500 can be implanted during a surgical procedure, such as an open-heart or other cardiac surgical procedure (e.g., a procedure in which one or more components of system 10 are also implanted). In embodiments in which leads 1500 are implanted for an extended period of time (e.g., without the intent of removal), the chronic nature of the implantation may result in strong adherence of leads 1500 to the outside of the heart wall, where the enhanced anchoring of leads 1500 can be more efficacious than temporary anchoring. In some embodiments, one or more external components of system 10 (e.g., one or more external devices, such as EPD 200 described herein) are configured to be reusable. Alternatively or additionally, one or more external portions of system 10 can be configured for single use (e.g., disposable). For example, an EPD 200 of system 10 can comprise a device that is configured to be used temporarily, such as when EPD 200 comprises a stimulator device configured as a temporary pacemaker (EPD 200_{PACE} shown), such as when the external device is also configured to deliver AF therapy, as described herein.

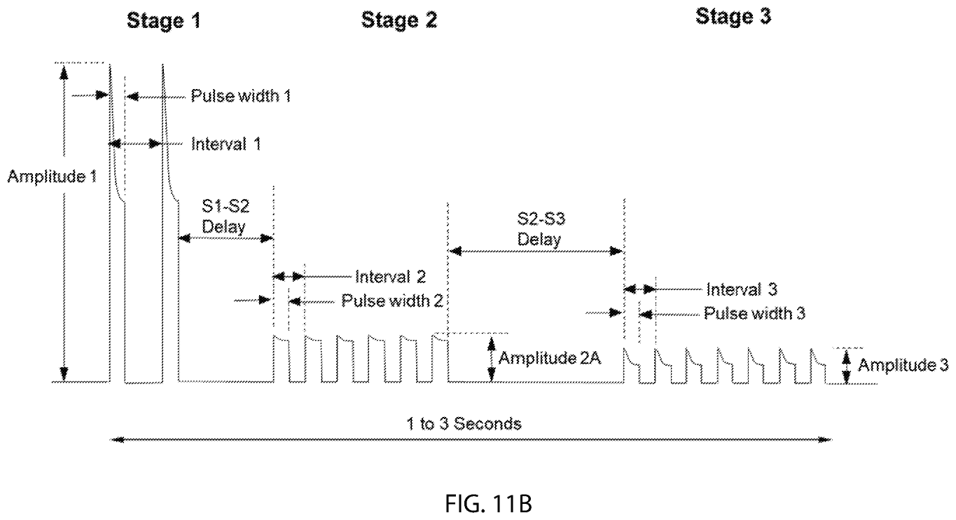

In some embodiments, system 10 comprises an external patient device 200 configured to convert a first waveform into a second waveform, EPD 200_{CVT} shown. For example, EPD 200_{CVT} can be configured to receive a first waveform (e.g., from a separate component of system 10 as described herein), such as a defibrillation pulse, and to convert the first waveform into a second waveform, such as a multi-pulse therapy waveform, also referred to as an MPT waveform. In some embodiments, an MPT waveform of the current inventive concepts comprises a biphasic waveform as shown in FIG. 11A and/or a monophasic waveform as shown in FIG. 11B. In some embodiments, an MPT waveform comprises multiple “stages”, such as the three stages shown in each of FIGS. 11A-B. Referring to the biphasic waveform of FIG. 11A, and the monophasic waveform of FIG. 11B, system 10 can be configured to provide such biphasic or monophasic waveforms, respectively, where the waveform comprises three stages (as shown), such as at least two stages. The levels for the signal parameters of the MPT waveform in the first stage can comprise: at least 1 pulse, such as at least 3 pulses, or at least 4 pulses; no more than 8 pulses; a positive pulse amplitude of at least 7.5V or at least 25V; a positive pulse amplitude of no more than 1230V, such as no more than 230V, no more than 150V, or no more than 100V; a pulse width of at least 1 ms, such as a pulse width of at least 4 ms, or at least 5 ms; a pulse width of no more than 20 ms; a time gap between pulses (interval 1 as shown in FIGS. 11A-B) of at least 10 ms, no more than 1000 ms, and/or between 30 ms and 35 ms; and combinations of one, two, or more of these. Referring specifically to the biphasic waveform of FIG. 11A, the negative pulse portion of the biphasic waveform can comprise the following levels for the signal parameters: a negative pulse with an amplitude that is at least the 10% of the magnitude of the amplitude of a positive pulse of the first stage; and/or a negative pulse with an amplitude that is no more than 90% of the magnitude of the amplitude of a positive pulse of the first stage.

The MPT waveform of the present inventive concepts can comprise multiple biphasic and/or monophasic pulses, as described herein. Monophasic pulses and pacing pulses can be delivered by system 10 to terminate cardiac tachyarrhythmias, such as by disrupting and extinguishing the rotational activity and drivers commonly known to initiate and sustain tachyarrhythmias. The MPT waveform delivered by system 10 accomplishes this treatment by using a unique waveform sequence which requires less total energy to terminate tachyarrhythmias than conventional single biphasic defibrillation therapy. The MPT waveforms mechanism of action creates virtual electrode polarization (VEP) at excitable heterogeneities within cardiac tissue, in close proximity to where rotational activity around a core or vortex works to anchor reentry. This VEP concept is a mechanism responsible for termination of atrial fibrillation (AF) and ventricular tachycardia (VT) at lower electrical energy thresholds, as demonstrated in experimental optical mapping studies in-vitro and in-vivo. Recent canine studies and human results have revealed that the therapy provided by the MPT waveform delivered by system 10 significantly lowers the energy required for atrial cardioversion and ventricular defibrillation. A first in man human AF trial conducted by applicant has found that the Stage 1 MPT waveform sequence significantly lowers the energy required for atrial cardioversion when compared to historical implantable device-based results with single biphasic shock. Mechanistically it is explained as: (Stage 1) unpinning of reentrant wave fronts that maintain AF, (Stage 2) preventing re-pinning of wave fronts to tissue heterogeneities, such as scars, and (Stage 3) extinguishes remaining wave fronts not self-extinguished. FIG. 11a provides a depiction of the MPT waveform when Stage 1 is programmed to biphasic delivery, and FIG. 11B when Stage 1 is programmed to monophasic exponential.

Referring back to both FIGS. 11A and 11B, either waveform (e.g., as provided by system 10) can comprise a second stage that is initiated (e.g., first pulse delivered): at least 20 ms from the end of the last pulse of the first stage, and/or no more than 1000 ms from the end of the last pulse of the first stage. The second stage can comprise at least 1 pulse, no more than 10 pulses, and/or approximately 6 pulses. Each pulse of the second stage can comprise a pulse width of at least 1 ms and/or no more than 20 ms. A time gap between pulses (interval 2 as shown in FIGS. 11A-B) can comprise a time of at least 2 ms, and/or no more than 500 ms. Each pulse of the second stage can comprise an amplitude of at least 0.5V or at least 3.5V, and/or an amplitude of no more than 10V or no more than 20V.

Also as shown in both FIGS. 11A and 11B, either waveform (e.g., as provided by system 10) can comprise a third stage that is initiated (e.g., first pulse delivered): at least 20 ms from the end of the last pulse of the second stage, and/or no more than 1000 ms from the end of the last pulse of the second stage. The third stage can comprise at least 1 pulse, no more than 30 pulses, and/or approximately 8 pulses. Each pulse of the third stage can comprise a pulse width of at least 0.2 ms and/or no more than 5 ms. A time gap between pulses (interval 3 as shown in FIGS. 11A-B) can comprise a time of at least 20 ms, and/or no more than 2000 ms. Each pulse of the third stage can comprise an amplitude of at least 0.25V or at least 1V, and/or an amplitude of no more than 10V or no more than 20V.

In some embodiments, EPD 200_{DEFIB} and/or EPD 200_{PACE} can be configured to deliver (e.g., without conversion of EPD 200_{CVT}) an MPT waveform as shown and described in reference to FIGS. 11A-B. In these embodiments, system 10 can include an EPD 200_{CVT} that provides control signals to the EPD 200_{DEFIB} and/or EPD 200_{PACE}, or system 10 can be void of the EPD 200_{CVT} component (e.g., EPD 200_{DEFIB} and/or EPD 200_{PACE} produce one or more of these MPT waveforms without any reliance on and/or assistance from, an EPD 200_{CVT}).

EPD 200_{CVT} can be configured to deliver the MPT waveform to the patient, such as via one or more electrodes 1510 of lead 1500 (e.g., a lead 1500 that is temporarily implanted in the patient to deliver a MPT waveform and/or other waveform received from EPD 200_{CVT}). EPD 200_{CVT} can be configured to operably attach to one, two, or more other external devices EPD 200 or other devices of system 10, such as one, two, or more devices configured to record signals from and/or deliver treatment energy to the patient. For example, EPD 200_{CVT} can be operably connected (e.g., at least electrically connected via one or more conduits and/or via a wireless connection) to an EPD 200 comprising and/or configured as an external defibrillator, EPD 200_{DEFIB} shown, and/or to an EPD 200 comprising and/or configured as an external pacemaker, EPD 200_{PACE} shown. EPD 200_{DEFIB} and EPD 200_{PACE} can be referred to singly or collectively herein as EPD 200_{DEFIB}. In some embodiments, EPD 200_{CVT} is configured to operate as a connecting hub, such that two or more external devices 200 can operably attach (e.g., via EPD 200_{CVT}) to a set of one or more leads 1500 that are attached to EPD 200_{CVT}. For example, EPD 200_{CVT} can allow for pass-through sensing (e.g., such that an attached device can sense one or more signals from one or more leads 1500 connected to EPD 200_{CVT}, and/or EPD 200_{CVT} can deliver stimulation energy received from an attached device to the patient via leads 1500. In some embodiments, EPD 200_{CVT} is configured to modify the stimulation energy received from the attached device before delivering the energy to the patient, such as is described herein (e.g., to regulate the received energy, and/or to convert the received energy into an MPT waveform). In some embodiments, EPD 200_{CVT} is configured to provide two or more forms of stimulation energy, such as an MPT waveform, SRT waveform, and/or traditional pacing waveforms. EPD 200_{CVT} can provide stimulation energy with or without the use of additional external devices (e.g., without the use of EPD 200_{DEFIB}). For example, system 10 can comprise a single EPD 200 that can be configured to provide the various post-operative therapy described herein via one or more leads 1500.

In some embodiments, EPD 200_{CVT} is configured to attach to a lead 1500 after a surgical procedure, such as to subsequently provide pacing (e.g., provide stimulation energy comprising one or more pacing waveforms, as described herein). EPD 200_{CVT} can be programmed (e.g., receive one or more instructions and/or other programming) by another device of system 10, such as an EPD 200_{DEFIB} that is operably connected (e.g., via a wired or wireless connection) to EPD 200_{CVT}. In some embodiments, EPD 200_{CVT} comprises a power source, such as a battery (e.g., power module 240 described herein). EPD 200_{CVT} can be configured to record and/or analyze one or more cardiac signals, as described herein.

In some embodiments, EPD 200_{CVT} is configured to be disposable (e.g., EPD 200_{CVT} is utilized a single time). In some embodiments, EPD 200_{CVT} has one or more sensing capabilities, for example, capabilities that are able to detect an occurrence of AF. In some embodiments, in response to identifying a positive detection of AF (e.g., after detecting an indication that the patient is undergoing AF), a request (e.g., a digital request) can be transmitted from EPD 200_{CVT} to a connected cardiac defibrillator and/or a cardiac pacemaker, such as EPD 200_{DEFIB} comprising a cardiac defibrillator and/or EPD 200_{PACE} comprising a cardiac pacemaker, respectively, and referred to singly or collectively herein as EPD 200_{DEFIB}. The request can be transmitted using a wired or wireless communication protocol. In some embodiments, the request contains an indication to provide energy (e.g., pacing and/or defibrillation energy) to EPD 200_{CVT}, where EPD 200_{CVT} converts the energy received into a low energy waveform (e.g., a low energy MPT waveform) that EPD 200_{CVT} delivers to a patient's heart via leads 1500. Alternatively, EPD 200_{CVT} sends a request to EPD 200_{DEFIB} to send a low energy waveform (e.g., a low energy MPT waveform) to the patient's heart (e.g., when EPD 200_{DEFIB} comprises a pacemaker and/or defibrillator that is also configured to deliver a low energy MPT waveform). In these embodiments, based on the specific characteristics of the patient and/or the detected AF condition (e.g., specific arrhythmia of the heart, age of patient, pre-existing conditions associated with the patient, and/or other patient specific characteristics), the request can identify the specific arrangement of therapy (e.g., number of pulses, rate of pulses, and/or intensity of pulses) to be delivered by EPD 200_{DEFIB} to effectively treat the specific occurrence of AF detected.

In some embodiments, EPD 200_{CVT} is configured to perform sensing and/or detection (e.g., detection of AF), and EPD 200_{DEFIB} comprises an external cardioverter configured to provide cardioversion therapy. In some embodiments, EPD 200_{CVT} is directly connected to one or more epicardial leads (e.g., leads 1500a-c shown). Each of leads 1500a-c can be temporarily and/or chronically placed during a surgical procedure (e.g., a cardiac surgery in which one or more adverse cardiac conditions are being treated). Leads 1500a and 1500b can be placed (e.g., a portion comprising electrodes 1510 can be placed) on the epicardial wall of the right atrium and the left atrium (respectively), and the leads 1500a and 1500b can be operably attached to EPD 200_{CVT}, such as to sense one or more cardiac signals and/or to deliver MPT waveform-based cardioversion therapy to convert AF to a normal sinus rhythm, as described herein. In some embodiments, lead 1500c is placed proximate the right ventricle and can be operably attached to EPD 200_{CVT} and/or EPD 200_{DEFIB}, such as to pace the ventricle to support and/or maintain sinus rhythm. For example, EPD 200_{CVT} can be configured to operably attach only to leads 1500a and 1500b for the treatment of AF. Lead 1500c can be directly attached to EPD 200_{DEFIB} and/or EPD 200_{PACE} (shown attached to EPD 200_{PACE} in FIG. 2), such as for the delivery of traditional stimulation (e.g., non-MPT waveform-based stimulation).

EPD 200_{CVT} can be configured to perform various functions, such as atrial sensing, classifying rhythm as POAF, and/or initiating a therapy request to EPD 200_{DEFIB} (e.g., when an AF condition is detected). Upon receiving the therapy request from EPD 200_{CVT}, EPD 200_{DEFIB} can deliver energy to EPD 200_{CVT}, and EPD 200_{CVT} can deliver one or more stimulation waveforms (e.g., low power MPT waveforms) to cardiac tissue via leads 1500, in an attempt to stabilize the heart rhythm. In some embodiments, EPD 200_{CVT} is configured to perform epicardial sensing (e.g., when electrodes 1510 of leads 1500 are positioned proximate the epicardium). In embodiments in which epicardial sensing is used, the latency issues known to be associated with surface sensing will be minimized, if not eliminated.

In some embodiments, EPD 200_{CVT} can include various electronic assemblies and/or componentry configured for cardiac signal sensing and/or analysis, such as when EPD 200_{CVT} comprises an AF detection algorithm (e.g., algorithm 215 not shown in FIG. 2 but described herein). Alternatively or additionally, EPD 200_{CVT} can include components and a protocol for communication with one or more other devices of system 10, such as EPD 200_{DEFIB} (e.g., a communication protocol of transceiver 220, not shown but described herein), such that cardiac signal analysis can be performed in the component in which EPD 200_{CVT} communicates. In some embodiments, EPD 200_{DEFIB} is configured to interface with (e.g., communicate with) EPD 200_{CVT}, for example, when EPD 200_{DEFIB} comprises communication componentry and an algorithm (e.g., algorithm 215) capable of communicating with EPD 200_{CVT}, analyzing the data provided in those communications, and responding by delivering an appropriate therapy to the patient.

In some embodiments, if EPD 200_{CVT} treatment fails (e.g., EPD 200_{CVT} is unsuccessful and/or otherwise ineffective and enters an associated alarm state), then a backup process for delivering therapy can be automatically initiated. For example, EPD 200_{DEFIB} can be configured to deliver the therapy directly to the heart in response to receiving an indication that EPD 200_{CVT} treatment has failed. In some embodiments, EPD 200_{DEFIB} only delivers the therapy to the heart in response to confirming that the patient has been properly medicated. For example, confirmation can be achieved dynamically, such as by outputting a request to an attending physician, and receiving confirmation from that physician that the patient is properly medicated. Alternatively or additionally, EPD 200_{DEFIB} can be in communication with a patient status system and/or drug delivery system, and can be configured to automatically confirm the patient medication status from the patient status and/or drug delivery system. In some embodiments, one or more devices of system 10 (e.g., EPD 200_{DEFIB} and/or EPD 200_{CVT}) are configured to perform additional “checks” (e.g., clinician confirmation checks and/or automated checks) prior to the delivery of the therapy (e.g., high energy therapy) provided by EPD 200_{DEFIB} and/or other devices of system 10.

In some embodiments, EPD 200_{CVT} can comprise a self-contained device (e.g., a multi-function device that is not reliant on information from a separate system 10 device). For example, EPD 200_{CVT} can be configured to perform each of sensing, detection, and delivery of one or more forms of therapy (e.g., cardiac stimulation and/or other cardiac therapy). In some embodiments, EPD 200_{CVT} can be operably attached to EPD 200_{DEFIB} such that EPD 200_{DEFIB} can deliver therapy to the patient if EPD 200_{CVT} fails to provide treatment due to an undetected AF event (e.g., as described herein).

In some embodiments, the initiation and delivery of therapy by EPD 200_{DEFIB} (e.g., EPD 200_{DEFIB} comprising an external cardioverter) may not be strictly binary in nature (e.g., EPD 200_{DEFIB} may not simply deliver therapy solely in response to detecting a fail signal from EPD 200_{CVT}). For example, EPD 200_{CVT} and/or EPD 200_{DEFIB} can be configured to analyze and weigh one or more factors prior to delivering therapy. EPD 200_{CVT} and/or EPD 200_{DEFIB} can be configured to delay delivery of therapy (e.g., therapy comprising one or more high energy pacing pulses) until a predetermined number of fail signals (e.g., at least three fail signals) are received from EPD 200_{CVT}, and/or if EPD 200_{CVT} has emitted a fail signal for a predetermined period of time (e.g., a time period of at least 5 seconds, at least 10 seconds, and/or at least 30 seconds). Such a time-dependent or multiple failure “check” can ensure that therapy is not delivered when EPD 200_{CVT} has only a momentary failure or other lapse of proper operation. Alternatively or additionally, EPD 200_{DEFIB} and/or EPD 200_{CVT} can be configured to confirm that a patient is appropriately medicated prior to delivering therapy, for example, as described herein. In some embodiments, EPD 200_{DEFIB} (e.g., EPD 200_{DEFIB} comprising an external cardioverter) and/or EPD 200_{CVT} can be configured to deliver therapy only during more serious AF events (e.g., during those AF events where the detected rhythm of the heart is greater than a predetermined threshold rhythm or other condition that surpasses a threshold).

In some embodiments, EPD 200_{DEFIB} and/or EPD 200_{CVT} can be configured to perform sensing of atrial signals, such as sensing via one or more surface electrodes, to detect AF events, such as to correspondingly send one or more command signals to EPD 200_{CVT} and/or EPD 200_{DEFIB}, respectively.

As shown in FIG. 2, EPD 200_{CVT} can be operably attached to a plurality of leads 1500, such as to leads 1500a-c shown, where each lead 1500 is attached to specific areas of the heart such that electrodes 1510 of each lead (electrodes 1510a-c shown) are contacting specific cardiac tissue. For example, lead 1500a can be attached to a location proximate the right atrium as shown (e.g., such that electrode 1510a is in contact with right atrial tissue), lead 1500b can be attached to a location proximate the left atrium as shown, and lead 1500c can be attached to a location proximate the right ventricle as shown. Leads 1500a-c can be coupled to EPD 200_{CVT}, as shown. In some embodiments, EPD 200_{CVT} is configured to operably attach to one or more other external devices, such as EPD 200_{DEFIB} and/or EPD 200_{PACE}, as shown. EPD 200_{CVT} can be configured to draw power and/or receive a signal (e.g., a pacing waveform drive signal) from EPD 200_{DEFIB} and/or EPD 200_{PACE}, such as via one or more electrical conduits, and/or via wireless power and/or data transfer arrangements (e.g., such as are described herein). EPD 200_{CVT} can be configured to record cardiac signals, and to analyze the recorded signals (e.g., via processing unit 210, not shown but described herein) to detect AF. Additionally or alternatively, EPD 200_{DEFIB} and/or EPD 200_{PACE} can be configured to record and/or analyze cardiac signals. Upon detection of an AF event, EPD 200_{CVT} can draw power from one or more of EPD 200_{DEFIB} and/or EPD 200_{PACE}, and to convert that energy into an MPT waveform, such as one or more MPT waveforms described herein. EPD 200_{CVT} can be configured to deliver the MPT waveforms (e.g., low-energy shocks) to the heart via electrodes 1510a-c of leads 1500a-c. In some embodiments, EPD 200_{DEFIB} and/or EPD 200_{PACE} are operably connected to one or more leads (e.g., a lead 1500) extending from the particular device to the cardiac tissue, such as the lead 1500c shown extending from EPD 200_{PACE}.

In some embodiments, EPD 200_{CVT} comprises a single use device. EPD 200_{CVT} can include one or more connectors configured to operably connect to one or more leads 1500.

Multi-pulse therapy (MPT) of the present inventive concepts comprises an energy delivery that applies pulsed electric fields to cardiac tissue between electrodes and/or coils (“electrodes” herein) that are strategically placed within and/or upon the patient's cardiac anatomy to optimize the distribution of field intensity across a given heart chamber and to minimize undesired collateral field effects, such as skeletal muscle contraction and/or pain. The MPT configuration can comprise up to three stages of energy delivery that each comprise a programable sequence of pulses, such as at least one pulse, or less than 4 pulses, or less than 10 pulses, with configurable amplitudes and/or widths that subsequently and incrementally deliver decreasing levels of energy. In some embodiments, the first stage comprises a single high-voltage pulse, such as at approximately 10% of the voltage of a full-strength shock provided by a conventional implantable cardioverter-defibrillator. The second stage delivers an incrementally lower (e.g., 30% to 70% lower) energy than the first stage, via three or more pulses, and/or less than 10 pulses. The third stage delivers a sequence of significantly lower amplitude pulses (e.g., pulses which are similar to conventional pacemaker pulses in amplitude and duration).

In some embodiments, EPD 200_{CVT} is configured to detect a QRS complex by analyzing ECG signals and/or EGM signals, and to deliver a first MPT pulse in a period of between 10 ms and 40 ms following the peak of the QRS complex. For example, EPD 200_{CVT} can comprise one or more leads, such as functional elements 299 shown that can be configured to record ECG and/or EGM signals. In some embodiments, EPD 200_{CVT} receives power from one or more batteries (e.g., one or more batteries of power module 240). EPD 200_{CVT} can include one or more buttons (e.g., one or more buttons of user interface 250), such as a button configured to deliver therapy to the patient, for example, to deliver therapy after the QRS complex after the activation of the button. In some embodiments, EPD 200_{CVT} is configured to allow up to ten MPT activation attempts. In some embodiments, EPD 200_{CVT} includes an on/off switch. In some embodiments, EPD 200_{CVT} includes a display, such as an LCD display, configured to notify the user of the state of EPD 200_{CVT}, for example, if the device is ready to deliver therapy and/or if the device is in a fault state (e.g., an alert state requiring a user correction before therapy can be delivered). EPD 200_{CVT} can be configured to record a series of MPT waveform deliveries performed by the device. In some embodiments, EPD 200_{CVT} is configured to function for up to seven days.

In some embodiments, leads 1500 comprise single use leads, such as sterile single use leads. Two leads 1500 can be packaged per sterile package, such as two leads 1500 packaged in a double tray. In some embodiments, leads 1500 are configured for an implantation duration of at least two days, five days, and/or seven days, and/or an implantation duration of no more than one week, no more than 1 month, and/or no more than 3 months. Leads 1500 can comprise flexible leads configured to conform to the epicardial wall of the left and/or right atrium, and/or to the pericardium above these structures. Leads 1500 can be configured to be implanted in less than five minutes, less than 10 minutes, and/or less than 30 minutes.

In some embodiments, leads 1500 are configured to be removed in a relatively simple procedure, such as a removal without surgical intervention (e.g., a removal from the patient when therapy is completed and/or when the patient is ready for discharge). Leads 1500 can be isodiametric and configured to prevent ingrowth and/or adhesion of tissue (e.g., for ease of withdrawal). In some embodiments, one or more portions of lead 1500 are configured to remain within the patient after removal of one or more other portions of lead 1500, and the remaining portions can be constructed of biocompatible materials and/or be bioabsorbable materials and configured to not pose an unacceptable risk in the chronic implant environment (e.g., avoid significant abrasion, cardiac tissue puncture, and/or MRI related risks).

Each lead 1500 is flexible enough to be placed anywhere on the surface of either atrium and/or either ventricle (e.g., to allow placement to achieve optimized vector of stimulation pathway to support optimized MPT waveform delivery, such as to reduce the energy requirements in terminating AF and/or other arrhythmia of the heart).

In some embodiments, electrodes 1510 of lead 1500 are of sufficient surface area and/or each lead 1500 can comprise a sufficient number of electrodes 1510 to achieve an appropriate surface area such that delivery of MPT produces impedances of less than 500 ohms, such as less than 100 ohms, and/or no more than 1000 ohms. For example, electrodes 1510 must be of sufficient surface area to safely deliver one or more MPT waveforms without resulting in damage to the lead 1500 materials and/or the tissue that contacts electrodes 1510. In some embodiments, delivery of an MPT waveform by system 10 results in a charge density of no more than 2.54 coulombs/mm², and/or no more than 3.0 coulombs/mm².

The electrodes should be placed in a manner that optimally distributes the field intensity across the heart chamber of interest, while reducing field effects to collateral structures. Generally, the gradient of the electric field that is sufficient to depolarize the tissue should optimally cover up to 50% of the RA and 80% of the LA, and minimally cover up to 30% of RA and 50% of LA.

Leads 1500 are configured to allow proper sensing and detecting of the peak of the QRS complex, and/or to deliver MPT waveforms. Each lead 1500 can comprise a connector that allows attachment to one or more other system 10 components, as described herein.

In some embodiments, one or more leads 1500 are configured to sense and detect the peak of the QRS complex of a patient's cardiac cycle (e.g., electrodes of leads 1500 record signals used by system 10 to identify the peak of the QRS complex). One or more leads 1500 can be configured to be placed on the ventricular wall and used for sensing of the QRS complex. One or more leads 1500 can be configured to sense and/or pace for bradycardia post-surgery.

While system 10 of FIG. 2 and otherwise herein is primarily described in treating atrial fibrillation (AF), various placements of leads 1500 and their associated electrodes can be used to treat other arrhythmias using system 10, such as when system 10 is configured to treat (e.g., via the delivery of one or more MPT waveforms as described herein): atrial tachycardia (AT); and/or ventricular arrhythmias, such as ventricular tachycardia (VT), supraventricular tachycardia (SVT), and/or ventricular fibrillation (VF). For example, EPD 200_{CVT} can be configured to receive energy comprising a first waveform from an EPD 200_{DEFIB} and/or an EPD 200_{PACE}, convert the received energy to energy comprising a different, second waveform (e.g., a MPT waveform), and deliver the energy comprising the different, second waveform to the patient's heart via electrodes 1510 of leads 1500, such that the second waveform treats (e.g., terminates) atrial fibrillation (AF), atrial tachycardia (AT), ventricular tachycardia (VT), supraventricular tachycardia (SVT), and/or ventricular fibrillation (VF) of the patient. For treating a particular type of arrhythmia, placement locations for leads 1500 (e.g., placement locations for the electrodes of one or more leads 1500) are chosen (e.g., by the clinician and/or an algorithm of system 10) to treat the particular arrhythmia. For example, the placement of leads 1500 is chosen to achieve an optimized vector (e.g., an efficacy-optimized vector) of a stimulation pathway produced by electrical energy delivered by the electrodes of leads 1500. In some embodiments, such as to treat a ventricular arrhythmia, a lead 1500 is positioned vertically along the lateral epicardium of the left ventricle, that can deliver energy to a lead 1500 placed internally in the right ventricle. Alternatively, a first lead 1500 can be placed anterolateral and a second lead 1500 placed posterolateral, where a combination of these leads is used with a third lead placed internally in the right ventricle. In some embodiments, one or more leads 1500 can be placed upon the epicardium of the right ventricle, and applied in combination with one or more leads 1500 placed on the epicardium of the left ventricle. In some embodiments, levels of signal parameters used by system 10 to treat an atrial arrhythmia are similar to the levels of signal parameters used by system 10 to treat a ventricular arrhythmia. These similarities can be due to the dimensional boundaries of the atria being reasonably comparable to the ventricles, despite the fact that the ventricular tissue is thicker (greater volume) than atrial tissue.

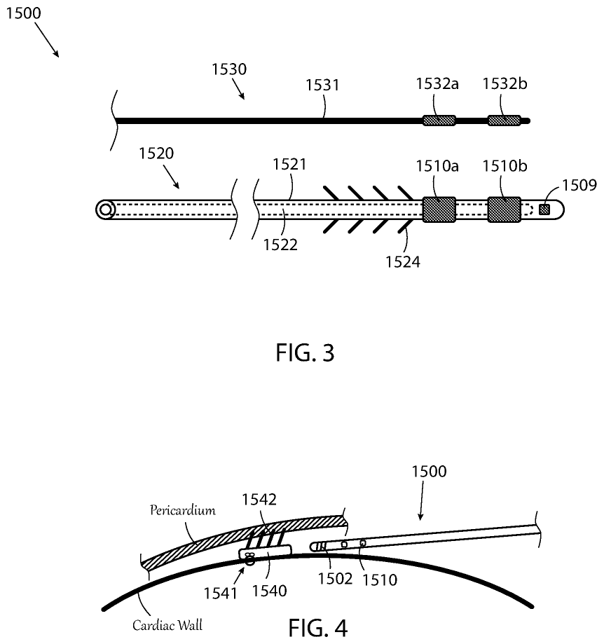

Referring now to FIG. 3, a side view of a cardiac lead is illustrated, consistent with the present inventive concepts. Leads 1500 of FIG. 3 can be of similar construction and arrangement as lead 1500 described in reference to FIG. 1 and/or otherwise herein. In some embodiments, lead 1500 comprises a two-part configuration, such as when lead 1500 comprises a first portion, sheath 1520, that is configured to be positioned near and fixedly attached to cardiac tissue. Sheath 1520 can comprise an elongate body, shaft 1521, with a lumen, lumen 1522, extending therethrough. The distal end of shaft 1521 can comprise a closed end, and can comprise a geometry for penetrating and/or grasping tissue, such as a barb-like and/or other penetrating and/or grasping-enabling geometry. Alternatively or additionally, the distal end of shaft 1521 can comprise an atraumatic, blunted geometry. One or more electrodes 1510 can be located on sheath 1520, such as two electrodes 1510a and 1510b located on the distal end of shaft 1521 as shown. In some embodiments, sheath 1520 can include one or more anchoring elements, anchors 1524 shown. Anchors 1524 can comprise one or more tines configured to engage tissue to fix the position of lead 1500 in the tissue. For example, electrodes 1510 can include one or more anchoring features, such as when anchors 1524 are integral to electrodes 1510. In some embodiments, anchors 1524 are configured to secure electrodes 1510 and/or lead 1500 relative to the cardiac tissue and/or the pericardium. One or more leads 1500 comprising a two-part configuration can also include a second portion, stylet 1530 shown. Stylet 1530 can comprise an elongate body, shaft 1531, that is slidingly received within lumen 1522 of sheath 1520. Stylet 1530 can include one or more electrical contacts, such as contacts 1532a and 1532b shown, located on shaft 1531. Contacts 1532a and 1532b can be located on shaft 1531 such that when shaft 1531 is positioned within lumen 1522 of sheath 1520, contacts 1532a and 1532b align with and make electrical connections with electrodes 1510a and 1510b, respectively. In some embodiments, the proximal end of stylet 1530 operably attaches to EPD 200_{CVT} and/or another device of system 10, such that contacts 1532a and 1532b are electrically connected to the device (e.g., such that pacing energy can be delivered by electrodes 1510 via contacts 1532). In some embodiments, stylet 1530 is configured to be slidingly received by and operably connected to sheath 1520 following a surgical procedure. For example, during a cardiac surgical procedure, one or more sheaths 1520 can be implanted for use in the delivery of AF treatment if POAF is detected. Approximately 10% to 50% of post-operative cardiac patients require AF therapy. If POAF is detected (e.g., detected by a device of system 10, such as EPD 200_{CVT} described herein), to deliver therapy stylet 1530 can be inserted into lumen 1522 of sheath 1520. For example, the proximal end of sheath 1520 can be positioned outside of the body (e.g., when lead 1500 is positioned through the skin of the patient), or the proximal end of sheath 1520 can be positioned beneath the surface of the skin (e.g., positioned by the clinician during a closing procedure of the cardiac surgery), such that stylet 1530 can be advanced percutaneously into lumen 1522. As described herein, stylet 1530 can operably connect EPD 200_{CVT} to electrodes 1510a, b such that EPD 200_{CVT} can deliver treatment energy to the tissue via electrodes 1510a,b.

In some embodiments, electrodes 1510 comprise cylindrical electrodes that surround a portion of shaft 1521. In some embodiments, lead 1500 can include one or more sensors or transducers, functional element 1509 shown. In some embodiments, electrode 1510 and/or functional element 1509 comprises one or more coils, such as elongate, cylindrical coils configured to distribute an electric field across a region of tissue (e.g., a region of tissue greater than that which would result from a similar drive signal applied to a similarly-sized electrode). In some embodiments, sheath 1520 is configured to traverse through tissue to place electrodes 1510 into the tissue. In some embodiments, a proximal portion of shaft 1521 is configured to be detached from the distal portion, such as after the proximal portion has been inserted into the tissue. For example, the proximal portion of shaft 1521 can be cut (e.g., cut by an operator of system 10, such as cut by a tool 90 comprising scissors or a knife) and discarded.

In some embodiments, lead 1500 is secured to a patient's heart tissue during a clinical procedure, such as a surgical procedure. Electrodes 1510 can be coupled to a wire (e.g., a microwire) that is configured to be coupled to an external therapy generator (e.g., EPD 200_{CVT} and/or another externally-positioned therapy-delivering device of system 10). For example, sheath 1520 can comprise one or more wires coupled to electrodes 1510. The one or more wires coupled to electrodes 1510 can be configured to operably connect to contacts 1532 of stylet 1530 (e.g., when contacts 1532 don't physically align with electrodes 1510 when stylet 1530 is fully inserted into lumen 1522). In some embodiments, stylet 1530 is configured to be introduced into the patient to make electrical contact with the wires coupled to electrodes 1510. For example, stylet 1530 can be introduced into the patient and slidingly received within lumen 1522 of sheath 1520. In some embodiments, one or more portions of sheath 1520, and/or electrodes 1510 can comprise materials that are bioabsorbable.

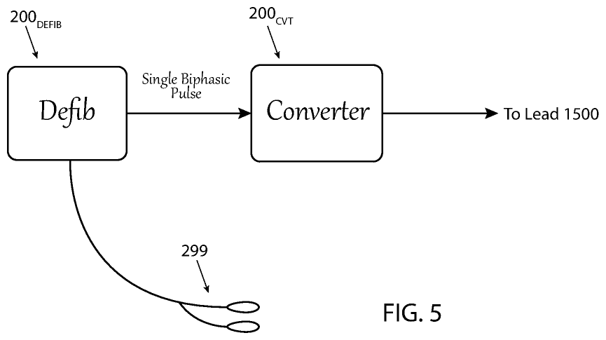

Referring now to FIG. 4, a side view of a cardiac lead and an anchoring clip is illustrated, consistent with the present inventive concepts. Lead 1500 and other components of system 10 shown in FIG. 4 can be of similar construction and arrangement to the similar components described in FIG. 1 and otherwise herein. Lead 1500 can comprise one or more removable anchoring elements, clip 1540 shown, such as an anchoring element configured to be secured to tissue, and to removably attach to a portion of lead 1500 (e.g., to temporarily secure lead 1500 to the tissue). In some embodiments, clip 1540 comprises a bioabsorbable material, such as a bioabsorbable polymer material. In some embodiments, clip 1540 is sutured (e.g., by a clinician during a surgical procedure) to a cardiac wall, such as to the outside surface of the atrium via suture 1541 shown. In some embodiments, suture 1541 comprises bioabsorbable suture. Alternatively or additionally, clip 1540 can include one or more securing elements, projections 1542 shown, which are configured to secure clip 1540 to the surface of the heart and/or to the pericardium. In some embodiments, clip 1540 is secured to the epicardium of the heart and not to the pericardium. In some embodiments, clip 1540 is secured to the pericardium and not the cardiac tissue (e.g., only secured to the pericardium). In some embodiments, one or more portions of clip 1540, such as projections 1542, pierce through one or more layers of the pericardium. Securing clip 1540 to the pericardium (e.g., and not the cardiac tissue) can reduce the tissue response of the cardiac tissue (e.g., tissue response caused by implanting clip 1540 and/or other portions of lead 1500) as the tissue around electrodes 1510 will not be damaged (e.g., the cardiac tissue is not damaged by suture 1541 and/or projections 1542). In some embodiments, lead 1500 is cylindrical in shape. Alternatively or additionally, at least a portion of lead 1500 can comprise a paddle-like shape, such as a shape that matches the curvature of the heart surface. Clip 1540 can be utilized for atrial and/or ventricular anchoring (e.g., to anchor lead 1500 to the atria and/or the ventricle, respectively).

In some embodiments, lead 1500 can include one or more mating features, connector 1502 shown, that engage with clip 1540 (e.g., via a threaded engagement, a bayonet engagement, and/or other reversible engagement mechanisms). In some embodiments, connector 1502 comprises a barb configured to engage clip 1540. In some embodiments, lead 1500 (e.g., at least a portion of lead 1500) is configured to be removed from the patient, for example, removed sometime after the procedure in which lead 1500 is implanted (e.g., at least 1, 3, 5 and/or 7 days after the implantation procedure). In some embodiments, at least a portion of lead 1500 is removed from the patient when the patient is discharged from the hospital, for example, stylet 1530 can be removed from sheath 1520 (each described in reference to FIG. 3 herein) and/or lead 1500 can otherwise be detached from an anchoring mechanism, such as clip 1540. For example, lead 1500 can be disengaged (e.g., unscrewed) from clip 1540, and extracted from the patient, leaving clip 1540 in place. Clip 1540 can be configured to resorb completely after lead 1500 has been removed. In some embodiments, clip 1540 comprises a length of less than 10 mm, or 5 mm. In some embodiments, clip 1540 is configured to hold electrodes 1510 of lead 1500 against the cardiac tissue.

In some embodiments, lead 1500 is configured to detach from clip 1540, such that lead 1500 can be removed from the patient without removing clip 1540. For example, lead 1500 can be configured to attach to and/or detach from clip 1540 via rotation (e.g., when threads are used to attach lead 1500 to clip 1540). In some embodiments, lead 1500 is configured to detach from clip 1540 under a tensile load, for example, when lead 1500 is attached to clip 1540 with a shear pin-like mechanism and/or a hook and loop attachment mechanism. In some embodiments, lead 1500 is temporarily attached to clip 1540 using a bioabsorbable connector, such as a connector configured to degrade over a period of time (e.g., approximately three days), such that lead 1500 can be removed after that time period. Lead 1500 can be attached to clip 1540 using a filament, such as a suture, that extends proximally along the length of lead 1500 (e.g., within a lumen of lead 1500), such that the proximal portion of the suture can be cut, releasing lead 1500 from clip 1540.

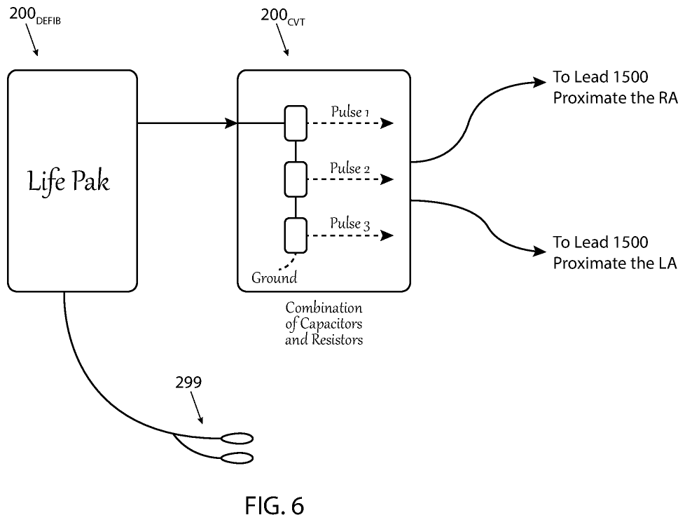

Referring now to FIG. 5, a schematic view of a system of multiple devices collectively configured to convert a defibrillation waveform from a first device to an MPT waveform to be delivered by a second device is illustrated, consistent with the present inventive concepts. EPD 200_{CVT} and other components of system 10 shown in FIG. 5 can be of similar construction and arrangement to similar components described in FIG. 1 and/or otherwise herein. In some embodiments, EPD 200_{CVT} is configured to receive a first waveform, such as a defibrillation waveform, and to convert the first waveform into a second waveform, such as an MPT waveform. EPD 200_{CVT} can be configured to deliver the MPT waveform to the patient, such as via one or more electrodes 1510 of lead 1500. As described herein, EPD 200_{DEFIB} can comprise a defibrillator (e.g., a commercially available defibrillator). EPD 200_{DEFIB} can be electrically coupled to EPD 200_{CVT}, such as via a wired and/or a wireless connection. In some embodiments, functional element 299 of EPD 200_{DEFIB} comprises one or more sensors, such as one or more electrodes configured to be positioned on the skin of the patient and to record cardiac signals (e.g., ECG signals).

In some embodiments, defibrillation therapy can be delivered directly to the patient from EPD 200_{CVT} (e.g., when defibrillation pulses are transmitted from EPD 200_{DEFIB} to EPD 200_{CVT}, and subsequently delivered to the patient via one or more electrodes 1510 of lead 1500). Alternatively or additionally, defibrillation pulses can be delivered directly to the patient from EPD 200_{DEFIB} (e.g., when EPD 200_{DEFIB} comprises one or more electrodes in contact with the patient for delivering defibrillation pulses to the patient, such as one or more electrodes 1510 of a lead 1500 operably connected to EPD 200_{DEFI}). In some embodiments, EPD 200_{DEFIB} is configured to output a biphasic pulse that is converted into an MPT waveform by EPD 200_{CVT}. In some embodiments, the MPT waveform is delivered to the patient by EPD 200_{CVT} with a delay (e.g., delivered after a predetermined and/or calculated time period). For example, a biphasic pulse can be triggered based on signals recorded by EPD 200_{DEFIB} (e.g., via electrodes of functional element 299), which can have a delay, for example, a delay determined by processing unit 210 of EPD 200_{DEFIB} (not shown but described herein), such as a delay that includes any processing latency plus additional delay time (e.g., a delay included to avoid delivering energy during the QRS complex). EPD 200_{CVT} can delay the delivery of the MPT waveform to effectively align with the cardiac cycle, for example, as described herein.

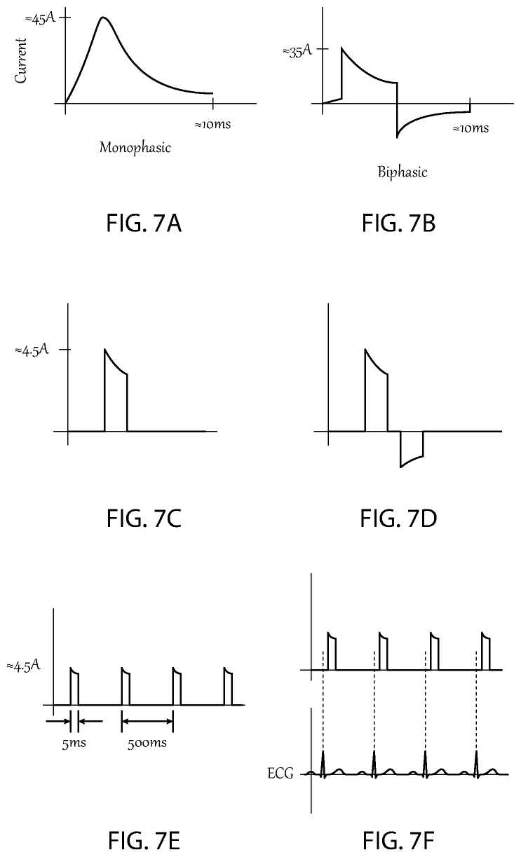

Referring additionally to FIG. 6, a schematic view of a converter device operably attached to a limited functionality device is illustrated, consistent with the present inventive concepts. In some embodiments, an EPD 200_{DEFIB} comprising a defibrillator or other cardiac pacing device can comprise a commercially and/or clinically available device (e.g., a device not produced and/or sold by the manufacturer of system 10, such as a Lifepak defibrillator device), such as a device with limited functionality that is improved when used in conjunction with EPD 200_{CVT}. In some embodiments, EPD 200_{CVT} is configured to operably attach to a limited functionality EPD 200_{DEFIB} (e.g., a Lifepak defibrillator) to perform synced cardioversion. For example, syncing can be achieved by monitoring cardiac signals and identifying the QRS complex. EPD 200_{CVT} can be configured to receive one or more defibrillation pulses from EPD 200_{DEFIB}, to store the energy of those pulses, and to deliver one or more stimulation pulses in a synchronized manner. For example, EPD 200_{CVT} can deliver the stimulation pulses after a 60 ms delay from the peak of the QRS complex. EPD 200_{CVT} can deliver two or more sequential pulses (e.g., three shown). In some embodiments, the sequential pulses are delivered at equal voltage. In some embodiments, additional energy received by EDP 200_{CVT} from EPD 200_{DEFIB} (e.g., energy not delivered to the patient) can be dissipated by EPD 200_{CVT}. EPD 200_{CVT} can comprise a combination of capacitors and resistors, for example, a combination of capacitors and resistors configured to dissipate any additional energy.

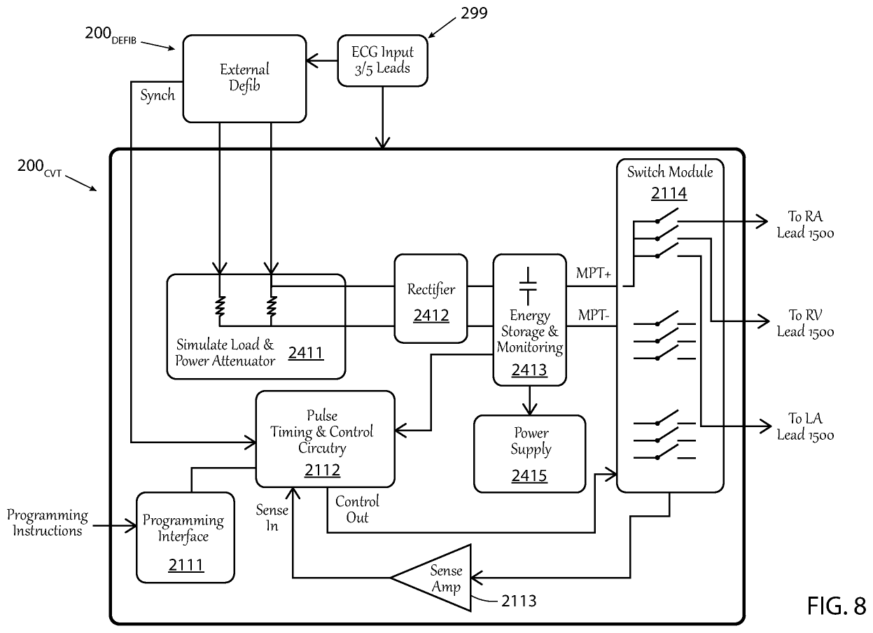

Referring now to FIGS. 7A-F, charts of various stimulation waveforms are illustrated, consistent with the present inventive concepts. As described herein, system 10 can comprise EPD 200_{CVT} configured to operably attach to EPD 200_{DEFIB} and to convert the output of EPD 200_{DEFIB} to an MPT waveform. In some embodiments, the MPT waveform can be delivered in multiple stages (e.g., three stages). In some embodiments, each stage of an MPT waveform can comprise a variable number of pulses, each with amplitudes, pulse widths, and/or other energy parameters that are determined by system 10. The output of EPD 200_{DEFIB} can comprise a monophasic waveform and/or a biphasic waveform, for example, as shown in FIGS. 7A and 7B, respectively. MPT waveforms can comprise low energy (e.g., lower energy than the output of EPD 200_{DEFIB}), multi-pulse defibrillation pulses. MPT waveforms can be monophasic and/or biphasic waveforms, for example, as shown in FIGS. 7C and 7D, respectively. MTP therapy can be configured to be delivered in a synchronized manner, such as when MPT pulses are delivered following the peak of a QRS complex, for example, as shown in FIG. 7F. In some embodiments, MPT pulses are synchronized based on cardiac signals recorded by EPD 200_{CVT} (e.g., signals recorded via one or more leads 1500 described herein). FIG. 7F shows four MPT stimulation pulses synchronized with the QRS complex of a recorded ECG signal.

FIG. 7A shows a monophasic pulse with an amplitude of current of approximately 45 A, and a duration of approximately 10 ms. The pulse shown in FIG. 7A can provide approximately 200 J into tissue (e.g., when the tissue provides a resistance of approximately 50Ω. FIG. 7B shows a biphasic pulse with an amplitude of current of approximately 35 A, and a duration of approximately 10 ms. The pulse shown in FIG. 7B can provide approximately 150 J into tissue (e.g., when the tissue provides a resistance of approximately 50Ω). FIG. 7E shows an example of an MPT waveform comprising low energy pulses (e.g., pulses of less than 10 J, such as approximately 5 J as shown, or less than 5 J, such as less than 3 J, less than 2 J, and/or less than or equal to 1 J). Each of the four pulses shown can comprise an amplitude of 4.5 A and a duration of 5 ms. The cycle length (e.g., the time between the start of a first pulse and the start of a following pulse) can be 500 ms, for example, when the MPT pulses are delivered asynchronously, at a rate of 120 beats per minute. When the MPT pulses are delivered synchronously, the cycle length can vary with the detected heart rate, as shown in FIG. 7F. Each of the pulses shown in FIG. 7E can provide approximately 5 J into tissue (e.g., when the tissue provides a resistance of approximately 50Ω). Collectively, the four pulses of FIG. 7E can provide approximately 20 J into tissue.

In some embodiments, EPD 200_{CVT} can be programmed to define the parameters of the MPT pulses to be delivered. For example, EPD 200_{CVT} can be programmed by clinician device 300 and/or other devices of system 10 described herein. Programmable (e.g., clinician variable) MPT pulse parameters can be selected from the group consisting of: synchronization status (e.g., whether or not the pulses are synchronized with recorded ECG signals); amplitude; pulse width; the type of pulse (e.g., monophasic or biphasic); timing of pulses; synchronization delay; and combinations of one, two, or more of these.

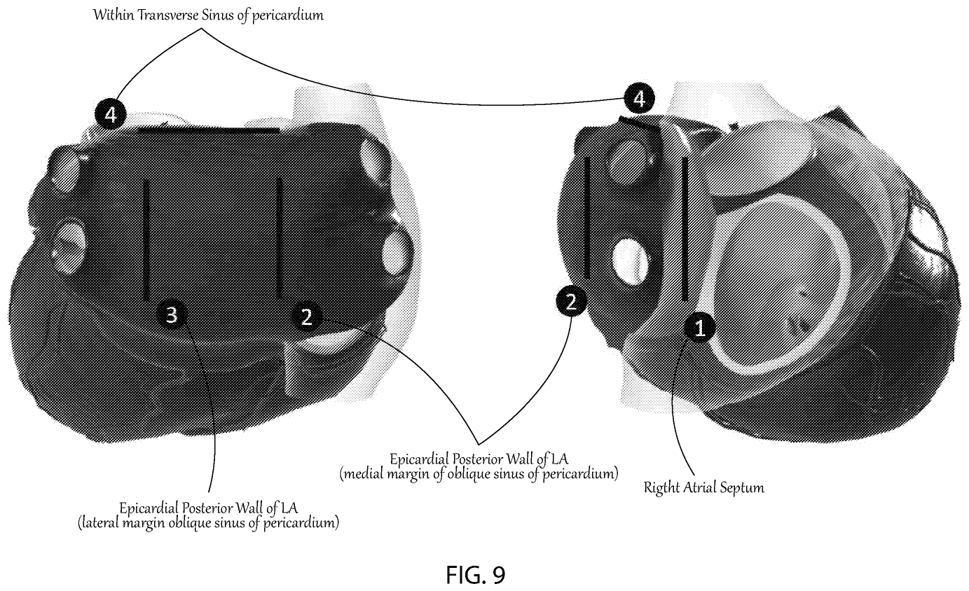

Referring now to FIG. 8, a schematic of a device configured to convert a waveform from a first device to a different waveform to be delivered by a second device is illustrated, consistent with the present inventive concepts. EPD 200_{CVT} can be configured to receive a waveform from a first device (e.g., a device configured as a standard pacemaker and/or defibrillator, such as EPD 200_{DEFIB} described herein) and convert the waveform to a MPT waveform of the present inventive concepts, such as to treat AF of the patient (e.g., convert an AF episode to a normal sinus rhythm). In the embodiment of FIG. 8, EPD 200_{CVT} can be operably attached to EPD 200_{DEFIB} as described herein. In some embodiments, EPD 200_{DEFIB} and/or EPD 200_{CVT} can be operably attached to a functional element of system 10 comprising one or more ECG leads, such as functional element 299 comprising three to ten ECG leads.

EPD 200_{CVT} can include module 2411 which can comprise a simulated load and power attenuator module configured to attenuate excess power received by EPD 200_{DEFIB}, for example, when MPT pulses delivered by EPD 200_{CVT} comprise less power than a defibrillation pulse received by EPD 200_{CVT} and converted to the MPT pulse, as described herein. EPD 200_{CVT} can include one or more signal rectifying components, rectifier 2412 shown, configured to allow EPD 200_{CVT} to convert positive, negative, and/or biphasic defibrillation pulses. EPD 200_{CVT} can include power supply 2415 (e.g., a power supply of power module 240 described herein), such as a power supply comprising a battery. EPD 200_{CVT} can include a module 2413 which can comprise an energy storage and monitoring module configured to store energy derived from input pulses from EPD 200_{DEFIB}. The module 2413 can be configured to monitor the available energy stored within the module and/or within power supply 2415 of EPD 200_{CVT}. In some embodiments, module 2413 is configured to recharge power supply 2415 with energy received from EPD 200_{DEFIB}. Processing unit 210 of EPD 200_{CVT} can comprise circuit 2112 which can comprise pulse timing and control circuitry configured to control the timing and/or parameters of MPT pulses delivered by EPD 200_{CVT}. In some embodiments, circuit 2112 receives a synchronization signal from EPD 200_{DEFIB}. EPD 200_{CVT} can include one or modules configured to multiplex various signal pathways, switching module 2114 shown, such as a module comprising one or more switches configured to connect the output of EPD 200_{CVT} to various leads 1500 as shown. Switching module 2114 can control the polarity of the pulses delivered by EPD 200_{CVT}, and/or switching module 2114 can be used to select which lead 1500 the pulses are delivered from. EPD 200_{CVT} can include one or more amplifiers, amplifier 2113 shown, such as a sensing amplifier configured to amplify one or more signals recorded by the leads 1500 operably attached to EPD 200_{CVT}. Signals amplified by amplifier 2113 can be analyzed by circuit 2112 to synchronize the pulses delivered by EPD 200_{CVT} to ECG signals. For example, ECG signals can be recorded by one or more leads, such as functional elements 299 shown that can be configured to record ECG and/or EGM signals. In some embodiments, circuit 2112 can detect a QRS complex or other cardiac waveform from recorded ECG signals. In some embodiments, EPD 200_{CVT} includes an interface for receiving programming or other instructions, programming interface 2111 shown, configured to receive programming and/or other information from one or more other devices of system 10, for example, as described herein.

In some embodiments, module 2411 comprises a pulse voltage charge pump and/or other voltage control components. In some embodiments, switching module 2114 includes a FET switch matrix and/or an H-Bridge. In some embodiments, circuit 2112 includes one or more defibrillation protection components, such as one or more components configured to protect circuit 2112 and/or other components of EPD 200_{CVT} from defibrillation pulses and/or other high voltage signals (e.g., high voltages signals that may enter EPD 200_{CVT} via leads 1500). In some embodiments amplifier 2113 comprises a filter, such as a power line filter, an anti-alias filter, and/or an EGM BP filter. In some embodiments, EPD 200_{CVT} comprises a user interface, such as user interface 250, not shown but described in reference to FIG. 1 and otherwise herein. User interface 250 can comprise an LCD screen, a keypad, and/or one or more visual indicators such as one or more LEDs. In some embodiments, EPD 200_{CVT} can include a transceiver, such as transceiver 220, not shown but described in reference to FIG. 1 and otherwise herein. Transceiver 220 can comprise a Bluetooth transceiver configured to communicate with one or more other devices of system 10.

In some embodiments, power module 240 comprises one or more components selected from the group consisting of: one or more batteries, such as four D Cell 1.5V alkaline batteries providing approximately 5 Ahr each; a power supply regulator; a pulse energy capacitor such as a 210 uF 500V film capacitor; a power supply regulator; and combinations of these.

In some embodiments, processing unit 210 comprises one or more components selected from the group consisting of: a pulse charge pump; an XFMR such as a Wurth 750032051; a controller such as an LT3750A DC-DC controller; an FET such as an Infineon IRF6644 FET NCH 100V 10 A controller; a diode such as a Vishay VS-6ESH06-M3; an Op Amp such as a FDBK amplifier; a microcontroller such as a Cypress CY8C4248LQI-BL563T PSoC ARM® Cortex®-M0 series microcontroller comprising a 32-Bit single-core m8 MHz 256 k flash 56-QFN with Bluetooth, LCD, ADC, and DAC; a crystal oscillator such as a 10 MHz ECS-100-18-5PX-TR; an N-CH FET switch such as a FQD7N30TM 300V 5.5 A NCH FET; a P-CH FET switch; such as a FQPF9P25 200V 6 A PCH FET; a defibrillation protection module, such as a Maxim MAX30034 10.3V 8UMax IC surge suppression module; one or more connectors, such as an EPI cable connector and/or a TE Connectivity 2097026-3 connector; one or more external cables, such as one or more sterile, single use external cables; an EGM vector FET switch, such as an analog switch; one or more amplifiers such as a TI INA122 instrument amplifier; and combinations of these.

In some embodiments, user interface 250 comprises one or more components selected from the group consisting of: an LCD display, such as a Displaytech 64128L FC BW3 display; a keypad, such as a membrane keypad; and combinations of these.

In some embodiments, antenna 225 is a multi-modality antenna, such as a 2.45 GHz Bluetooth, ISM, Wi-Fi, Zigbee™ chip antenna.

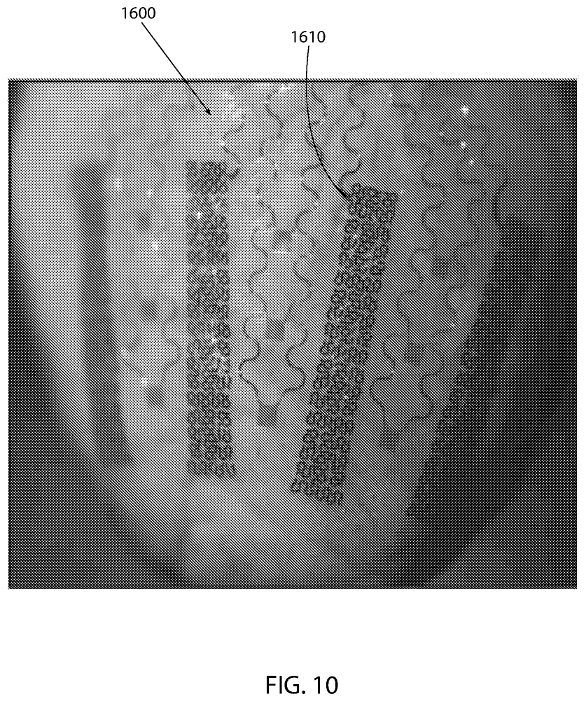

Referring now to FIG. 9, anatomic views of a heart showing various pacing configurations are illustrated, consistent with the present inventive concepts. System 10 can be configured to apply sequential and/or simultaneous energy deliveries across two or more segments of electrodes and/or coils (“electrodes” herein) placed in two or more of locations 1, 2, 3, and/or 4 of FIG. 9. For example, system 10 can be configured to apply energy between locations 2 and 3 to control (e.g., pace or otherwise effect) the posterior wall of the heart, which can be followed by energy delivery between locations 1 and 3, or locations 1 and 4, to control the anterior wall of the heart.

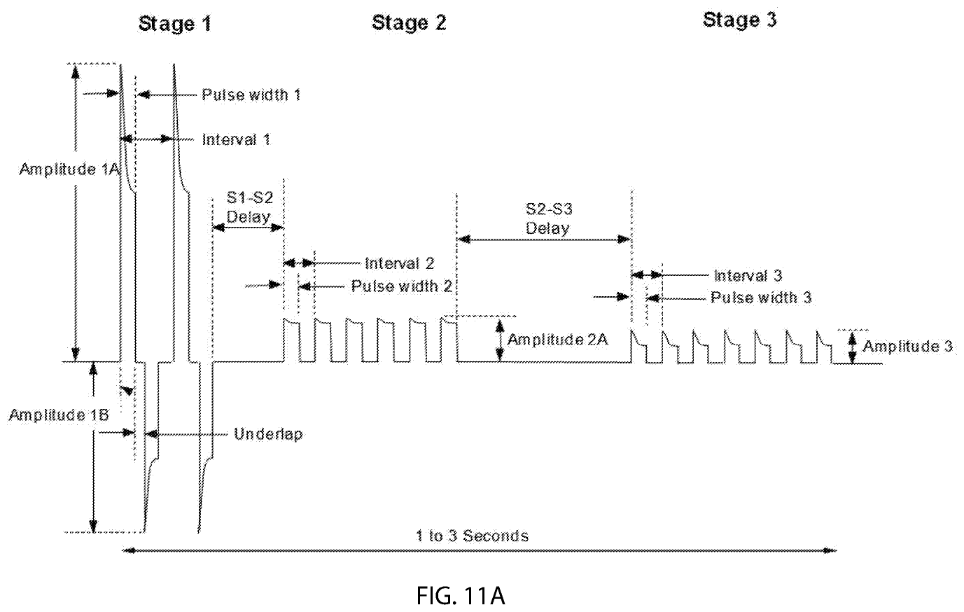

Referring now to FIG. 10, a photograph of an electrode patch is illustrated, consistent with the present inventive concepts. In some embodiments, system 10 comprises one or more arrays of functional elements (e.g., sensors and/or transducers), patch array 1600, comprising one, two or more sensor and/or transducer elements such as electrodes, electrodes 1610. In some embodiments, array 1600, electrodes 1610, and other components of system 10 described in reference to FIG. 10 can be of similar construction and arrangement to electrode array 110, electrodes 111, and/or other similar components described in reference to FIG. 1 and otherwise herein. Alternatively or additionally, electrodes 1610 can be of similar construction and arrangement to electrodes 1510 of lead 1500 described in reference to FIG. 1 and otherwise herein. For example, patch array 1600 can be located at the distal end of lead 1500.

In some embodiments, patch array 1600 comprises a fractal boundary, as shown. For example, one or more electrodes 1610 of array 1600 can comprise a fractal-like geometry, as shown, such as a fractal-like geometry that maximizes edge length to reduce electrode interface impedance. In some embodiments, patch array 1600 can be operably attached to a device of system 10, such as ID 100, EPD 200, and/or EPD 200_{CVT} described herein, such that electrodes 1610 can be used by the device to sense one or more cardiac signals and/or to deliver therapeutic energy, as described herein.

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the inventive concepts, which is defined in the accompanying claims.

1. A system for providing post operative treatment of atrial fibrillation to a patient, the system comprising:
one or more leads, each lead comprising one or more electrodes for delivering energy to the heart of the patient;
an energy delivery device for providing energy comprising a first waveform; and
a converter device electrically connected to the energy delivery device, the converter device configured to:
receive the energy comprising the first waveform from the energy delivery device;
convert the energy comprising the first waveform into energy comprising a second waveform; and
deliver the energy comprising the second waveform to the patient's heart via the one or more electrodes of each of the one or more leads,
wherein the energy comprising the second waveform delivered by the converter treats atrial fibrillation of the patient.

2. (canceled)
